(19) United States
(12) Patent Application Publication
(10) Pub. No.: US 2024/0108480 A1
(43) Pub. Date: Apr. 4, 2024

(54) CLUTCH SYSTEM FOR A POWERED PROSTHETIC DEVICE

(71) Applicant: Point Designs LLC, Lafayette, CO (US)

(72) Inventors: Benjamin Pulver, Louisville, CO (US); Levin Sliker, Boulder, CO (US); Stephen Huddle, Thornton, CO (US); Richard Weir, Lafayette, CO (US); Jacob Segil, Boulder, CO (US); Barathwaj Murali, Broomfield, CO (US); Andrew Taylor, Denver, CO (US); Tom Noe, The Hauge (NL)

(21) Appl. No.: 18/375,953

(22) Filed: Oct. 2, 2023

Related U.S. Application Data
(60) Provisional application No. 63/412,828, filed on Oct. 3, 2022.

(51) Int. Cl. A61F 2/58; A61F 2/70

(52) U.S. Cl. CPC A61F 2/586; A61F 2/70; A61F 2002/6836; A61F 2002/701

(57) ABSTRACT

Embodiments are directed to a prosthetic finger that includes a track that couples to a hand, a first phalange coupled to the track at a first joint, a second phalange coupled to the first phalange at a second joint, a third phalange coupled to the second phalange at a third joint, and a motor positioned at least partially within the second phalange. The prosthetic finger can include a first linkage positioned within the first phalange and configured to cause the first phalange to move relative to the knuckle in response to a motor output and cause the second phalange to move relative to the first phalange in response to the motor output. The prosthetic finger can include a second linkage coupling the third phalange to the second phalange and configured to cause the third phalange to move relative to the second phalange in response to the motor output.

### CROSS-REFERENCE TO RELATED APPLICATION

This application is a nonprovisional of, and claims the benefit under 35 U.S.C. § 119(e) of, U.S. Provisional Patent Application No. 63/412,828 filed Oct. 3, 2022, the contents of which are incorporated herein by reference as if fully disclosed herein.

### FIELD

The described embodiments relate generally to prosthetic devices. More particularly, the present embodiments relate to prosthetic devices for the hand.

### BACKGROUND

Finger or partial hand prostheses can be used to restore function and/or appearance of a missing finger or thumb. A prosthetic socket may be used to attach a finger and/or hand prosthesis to a user. A socket may be custom made to fit over a portion of a user's limb and the finger or hand prosthesis may be attached to the socket. In cases where a user may be missing an individual finger, the prosthetic socket may attach to the user's hand at the missing finger and an individual finger prosthesis may attach to a socket. In other cases, a user may be missing multiple digits, a hand, and/or a portion of their limb. The prosthesis may include a hand portion and one or more prosthetic digits may attach to the hand portion.

Traditional finger prostheses position the prosthesis in a fixed location with respect to other fingers of the hand. These types of prostheses may have significantly limited ranges of motion as compared to a natural finger. A user may desire a prosthetic device that functions more similar to a natural finger.

### SUMMARY

Embodiments described herein are directed to a prosthetic finger that includes a track that couples to a hand, a first phalange coupled to the track at a first joint, a second phalange coupled to the first phalange at a second joint, and a third phalange coupled to the second phalange at a third joint. A motor can be positioned at least partially within the second phalange. The prosthetic finger can include a first linkage positioned at least partially within the first phalange and configured to cause the first phalange to move with respect to the track in response to a motor output, and cause the second phalange to move with respect to the first phalange in response to the motor output. A second linkage can couple the third phalange to the first phalange and be configured to cause the third phalange to move with respect to the second phalange in response to the motor output.

Embodiments described herein are directed to a prosthetic finger that includes a first phalange that couples to a hand, a second phalange that couples to the first phalange at a joint, and a motor positioned at least partially within the second phalange. The prosthetic finger can include a cycloidal gear system that includes an input gear operably coupled to the motor and an output shaft coupled to the joint; wherein, in response to an output from the motor, the cycloidal gear system causes the second phalange to move with respect to the first phalange.

Embodiments described herein also include a prosthetic finger that includes a first phalange that couples to a hand, and a second phalange coupled to the first phalange at a joint. The second phalange can include a body portion, a motor positioned at least partially within the body portion, and a gear housing coupled to the body portion and comprising a gear system operably coupled to the motor. The prosthetic finger can include a clutch positioned at the joint and configured to cause an output of the gear system to move the first phalange with respect to the second phalange in response to a force on the clutch below a threshold, and cause the gear system to statically couple with respect to the first phalange and prevent movement of the first phalange with respect to the second phalange in response to the force on the clutch meeting or exceeding the threshold.

### BRIEF DESCRIPTION OF THE DRAWINGS

The disclosure will be readily understood by the following detailed description in conjunction with the accompanying drawings, wherein like reference numerals designate like structural elements, and in which:

FIGS. 1A and 1B show examples of a powered prosthetic finger attached to a hand of a user;

FIGS. 2A-2B show examples of a powered prosthetic finger;

FIGS. 3A-3C show examples of a track and a first phalange of a powered prosthetic finger;

FIGS. 4A-4B show examples of a second phalange of a powered prosthetic finger;

FIGS. 5A and 5B show examples of the linkage system and internal portion of the first phalange in different flexion states;

FIGS. 6A-6C show examples of movement of a powered prosthetic finger from an extended position to a flexed position;

FIGS. 7A-7D show an example of a powered prosthetic finger with a single link bar coupled to the track and the middle phalange;

FIG. 8 shows an example of a powered prostatic finger that has two phalanges;

FIG. 9 shows an example of a drive system output on the prosthetic finger;

FIG. 10A shows an example of a drive system for a prosthetic finger;

FIG. 10B shows an example of a gear system for a prosthetic finger;

FIG. 11 shows an exploded view of the gear system shown in FIGS. 10A and 10B;

FIG. 12 shows an exploded view of an example gear system;

FIG. 13A shows an assembled view of the first gear stage of a gear system for a prosthetic finger;

FIG. 13B shows an exploded view of the first gear stage shown in FIG. 12A;

FIG. 14A shows an assembled view of the second gear stage of a gear system for a prosthetic finger;

FIG. 14B shows an exploded view of the second gear stage shown in FIG. 13A;

FIG. 15A shows an example of a drive system for a prosthetic finger;

FIG. 15B shows an example of a drive system for a prosthetic finger;

FIG. 16 show an example of a gear system for a prosthetic finger;

FIG. 17A shows a first exploded view of a gear system for a prosthetic finger;

FIG. 17B shows a second exploded view of a portion of the gear system shown in FIG. 17A;

FIG. 18 shows a cross-sectional view of the drive system shown FIG. 15A;

FIG. 19A shows an example carrier housing for the gear systems described herein;

FIG. 19B shows an example of an assembly of a carrier housing and cycloidal gears;

FIG. 19C shows an example of an assembly of a first carrier housing portion to a second carrier housing portion;

FIGS. 20A and 20B show an example of output shaft configurations that can be used to control engagement of a clutch system;

FIGS. 21A-21D show additional examples of cycloidal gear configurations that can be used in a powered prosthetic finger;

FIG. 22 shows an example clutch system for a powered prosthetic finger;

FIG. 23A shows a perspective view of the clutch system with a first portion of the first phalange removed;

FIG. 23B shows a perspective view of the clutch system with a second portion of the first phalange removed;

FIG. 24 shows a perspective view of the clutch system with the second portion of the first phalange removed;

FIG. 25 shows a perspective view of the clutch system with a first portion of the first phalange removed;

FIGS. 26A and 26B show an example operation of the clutch system;

FIGS. 27A and 27B show an example of an output shaft configuration that can be used to control engagement of the clutch system, such as the clutch system of FIG. 14;

FIGS. 28A and 28B show an example clutch system for a prosthetic finger; and

FIGS. 29A and 29B show an example operation of the example clutch system shown in FIGS. 28A and 28B.

FIGS. 30A-30C show an example of a prosthetic finger that includes a cantilevered clutch mechanism;

FIGS. 31A and 31B show cross-sectional views of an example of the cantilevered clutch mechanism shown in FIGS. 30A-30C;

FIGS. 32A and 32B show cross-sectional views of an example of the cantilevered clutch mechanism shown in FIGS. 30A-30C;

FIGS. 33A and 33B show an example of a prosthetic finger that includes a slotted clutch mechanism;

FIGS. 34A and 34B show example operation of the slotted clutch mechanism shown in FIGS. 33A and 33B;

FIGS. 35A-3C show an example of a prosthetic finger that includes a cantilevered clutch mechanism;

FIGS. 36A and 36B show an example of a prosthetic finger that includes a cantilevered clutch mechanism incorporating a linkage system; and

FIGS. 37A and 37B show an example of a prosthetic finger that includes a cantilevered clutch mechanism.

It should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments described herein are directed to a powered prosthetic finger (also referend to as a “prosthetic finger”) that includes a motor and a gear system that function together to drive movement of the prosthetic finger. The prosthetic finger may include multiple phalanges that are coupled together to emulate the motion of a natural finger. Movement of the phalanges with respect to each other and with respect to a user's hand may be controlled by a linkage system. As the motor is driven, motion of the motor, via the gear system, may cause the phalanges to move in a path defined by the coupling of the phalanges and the linkage system. Driving the motor in a first direction causes the phalanges to move in flexion which emulates a flexion motion of a natural digit. Driving the motor in a second direction causes the phalanges to move in extension which emulates an extension motion of a natural digit. Maintaining the motor in a static state can cause the phalanges to remain stationary or locked, which may emulate a gripping motion of a natural digit.

The prosthetic finger may be controlled using myoelectric signals of the user. For example, myoelectric sensors may be attached to a user and outputs from the myoelectric sensors may be used to determine an intended motion for the prosthetic finger. A control system may use the myoelectric outputs to generate control signals for the prosthetic finger. In some cases, the control signals may be motor control signals which control operating parameters of the motor. The controlled operating parameters may include a direction of rotation, rotational speed, driving duration, speed profile, and/or any other suitable parameters of the motor.

In cases where a user is missing less than all of their natural digits, one or more prosthetic fingers may couple to a prosthetic socket(s) that is/are positioned on the user's natural hand. In cases where a user has a prosthetic hand, one or more prosthetic fingers may be coupled to the prosthetic hand. As used herein the term “hand” may be used to refer to a user's natural hand or a prosthetic hand.

The prosthetic finger can include a track that attaches to a hand of a user. A first (proximal) phalange may be coupled to the track at a first joint, a second (middle) phalange may be coupled to the first phalange at a second joint, and a third (distal) phalange may be coupled to the second phalange at a third joint. In some cases, the first joint may correspond to the metacarpophalangeal (MCP) joint of a hand, the second joint may correspond to the proximal interphalangeal (PIP) joint of a hand, and the third joint may correspond to the distal interphalangeal (DIP) joint of the hand.

The exemplary prosthetic fingers are described herein primarily in the context of three phalange prosthetic devices. However, the concepts described herein can be applied to prosthetics with greater or fewer phalanges. For example, the concepts described herein may be applied to a prosthetic finger having two phalanges, which may be used for partial finger amputations. In some cases, a two phalange prosthetic finger may include a track that attaches to a proximal phalange of a user, for example using a prosthetic socket. The first phalange of the two phalange prosthetic finger may correspond to a middle phalange or a proximal phalange and the second phalange may correspond to a distal phalange or a combined middle and distal phalange (e.g., middle and distal phalange do not move with respect to each other. In these cases, a first joint defined by the track and the base of the first phalange may correspond to a PIP joint and a second joint defined by the coupling of the first phalange and the second phalange may correspond to the DIP joint. In some cases, a two (or single phalange) device can be applied in cases where the digit includes a thumb of the user.

In some cases, one or more phalanges may be rigidly coupled together and may be referred to as a single phalange. For example, a prosthetic finger may include a phalange that has a medial phalange and distal phalange that are rigidly coupled together (e.g., there is no relative movement between the distal phalange and the medial phalange). In these cases, the distal and medial phalanges may be distinguished by surface features and/or other features. For example, a distal portion of the single phalange may by angled with respect to a medial portion of the phalange. Additionally or alternatively, the distal portion of the phalange may include different material, such as a polymer that can aid gripping of objects and/or other actions of the user. In these embodiments the prosthetic finger may be referred to as a two phalange system with a single phalange incorporating features that correspond to two natural phalanges (e.g., a single mechanical phalange has features that correspond to both a medial and distal phalange).

The first phalange may be configured to move along the track in a first direction that corresponds to flexion of the prosthetic finger and a second direction that corresponds to extension of the prosthetic finger. In some cases, the track can include one or more rails and the first phalange can include one or more retention features that couple the first phalange to the track and allow the first phalange to move along the track in the first and second directions. The track may be curved, which may cause the first phalange to move along a curved profile during flexion and extension movements. The combination of the curved track and the positioning of the track on the prosthetic socket or prosthetic hand can locate the center of motion within the hand or at a position that is remote from the mounting location of the track. Such configurations can result in an externally mounted prosthetic device that is able to impart similar motion to the prosthetic digit as an internal joint of a natural digit. Accordingly, the shape of the track and its orientation on the prosthetic socket can be configured such that the movement of the prosthetic digit can achieve similar motion to the motion of a natural digit.

The second phalange may be rotationally coupled to the first phalange and rotate about the first phalange to define the second joint. A distal end of the first phalange may be coupled to the proximal end of the second phalange. The first and second phalanges may define a first axis of rotation that causes the second phalange to rotate in the first direction. Accordingly, as the first phalange moves along the track in a first direction, the second phalange may rotate about the first phalange in the first direction. The motion of the first and second phalange in the first direction may correspond to flexion of the prosthetic finger. As the first phalange moves along the track in the second direction, the second phalange may rotate about the first phalange in the second direction, which may correspond to extension of the prosthetic finger. The combined motion of the first phalange along the track and the rotation of the second phalange with respect to the first phalange may cause the first phalange to move toward the axis of rotation, which can emulate a gripping/grasping motion that is performed by a natural finger.

A third phalange may be rotationally coupled to the second phalange and rotate about the second phalange to define the third joint. A distal end of the second phalange may be coupled to a proximal end of the third phalange. The second and third phalanges may define a second axis of rotation that causes the third phalange to rotate in the first direction. Accordingly, as the first phalange moves along the track, the third phalange may rotate about the second phalange in the first direction, which may correspond to flexion of the prosthetic finger. Movement of the first phalange in the second direction may cause the third phalange to rotate about the second phalange in the second direction, which may correspond to extension of the prosthetic finger. The combined motion of the first phalange along the track, the rotation of the second phalange with respect to the first phalange, and rotation of the third phalange with respect to the second phalange may cause the first and second phalanges to move toward the track, which can emulate a gripping/grasping motion that is performed by a natural finger.

The prosthetic finger can include a linkage system which defines the relative motion between the track, the first phalange, the second phalange, and the third phalange. In some cases, the linkage system can include a first linkage set that defines the relative motions of the track, the first phalange, and the second phalange. The first linkage set may cause the first phalange to move along the track and the second phalange to rotate about the first phalange in response to a motor output. The first linkage set can include a three-linkage system that is positioned at least partially within an interior cavity defined by the first phalange. The three-linkage system may include a first link bar having a first end coupled to the track, a second link bar having a first end coupled to the first phalange, and a third link bar having a first end coupled to the second phalange. The second ends of each of the first, second, and third link bars may be coupled together within the interior cavity of the first phalange. The use of a three-linkage system may help maintain the linkage system within the interior cavity of the first phalange as the prosthetic finger moves in flexion and/or extension, as described herein. For example, as the prosthetic finger moves in flexion, the majority of the first linkage set may remain within the first phalange throughout the movement profile. Accordingly, the multi-linkage system may reduce interference of the linkages within the grasping space, which may occur with other linkage configurations.

The linkage system can include a second linkage set that defines the relative motion of the second and third phalanges. The second linkage set can include one or more link bars that include a first end that is coupled to the second joint (e.g., PIP joint of the prosthetic finger) and a second end that is coupled to the third phalange. As the first and second phalanges move, the second linkage set can cause the third phalange to rotate about the second phalange as described herein.

The motor and gear system may be positioned at the second phalange. For example, the motor may be positioned at least partially within the second phalange and include an output gear that rotates to provide a motor output. A length dimension of the motor may extend along a length dimension of the second phalange and a base of the motor may be positioned proximate a distal end of the second phalange and the output gear may be oriented toward the second joint (e.g., the PIP joint). The gear system may couple to the second phalange and include an input gear that couples to the output gear of the motor. The gear system can include an output shaft that couples to the second joint (e.g., the PIP joint) and causes flexion and extension of the prosthetic finger.

The gear system may include a cycloidal gear system that translates a rotational output of the motor to provide a force that causes movement of the prosthetic finger. For example, the rotational output of the motor can apply a torque that causes rotational of the phalanges with respect to each other. The gear system may increase the force output (e.g., torque) of the motor to move the finger. In some cases, the gear system can include a multi-stage gear cycloidal gear. Each gear stage may provide a reduction in the output speed from the motor and an increase in the output force. The output of a first stage may be input to a second stage gear and the second stage may cause the output shaft to rotate thereby moving the finger.

In some cases, the prosthetic finger can include a clutch system, which may be used to protect the gear system from damage, for example, due to force exerted on the prosthetic finger during a gripping operation. The clutch system may lock the second phalange and the first phalange together, thereby preventing movement of the prosthetic finger. The locking action of the clutch system may cause forces on the finger to be primarily applied to the phalange structures and clutch structures, which may reduce the forces applied to the gear system. For this reason, the clutch system may help protect the gear system components and/or increase forces that can be applied to the prosthetic finger, such as forces during a gripping operation. Accordingly, the clutch system may increase an amount of static grip force of the prosthetic finger thereby allowing a user to pick up heavier objects without risking damaging the gear system.

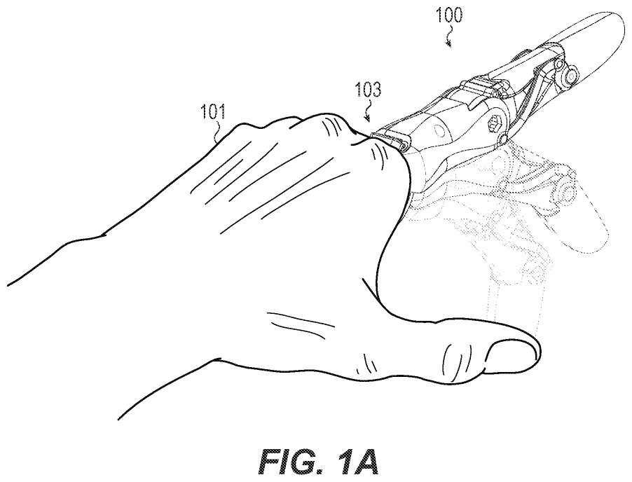

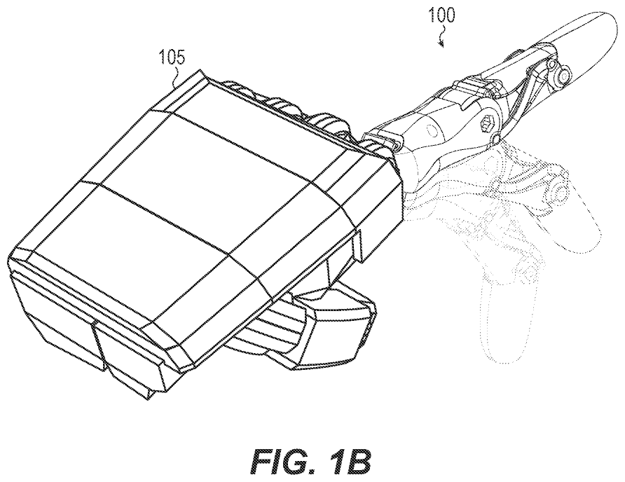

These and other embodiments are discussed below with reference to FIGS. 1A-37B. However, those skilled in the art will readily appreciate that the detailed description given herein with respect to these Figures is for explanatory purposes only and should not be construed as limiting.

FIG. 1A shows an example of a powered prosthetic finger 100 attached to a hand 101 of a user. A socket 103 may be attached to the hand 101 and the prosthetic finger 100 can attach to the socket 103. The socket 103 may be custom made for a user and include an interior portion that conforms to the anatomical features of a user's hand and/or limb. The socket 103 may include one or more layers including one or more rigid layers. In some cases, the prosthetic finger 100 may be attached to the socket 103 using one or more fasteners, such as screws that fasten the prosthetic finger 100 to the socket 103. In other cases, the socket may include fastening features, such as anchors or nuts, that can interface with fastening features on the prosthetic finger 100. The anchors, nuts or other fasteners may be integrated into the socket 103 as it is being built for the user and/or attached to the prosthetic socket after it has been built for the user.

The prosthetic finger 100 may have a defined range of motion and move in flexion and extension directions through the range of motion. The range of motion of the prosthetic finger 100 can be configured to emulate the motions of a natural finger. For example, the prosthetic finger 100 may move in flexion, in which the base of the prosthetic finger 100 moves toward the palm, and move in extension, in which the base of the prosthetic finger 100 moves away from the palm. Flexion movements may be used to grip an object, make a fist or emulate other functions of the hand and extension movements may be used to release an object or to otherwise open up the finger and/or hand. The range of the motion of the prosthetic finger 100 may span from a full extension position to a full flexion position. The full extension position may correspond to each of the phalanges substantially aligning along a length dimension and the full flexion position may correspond to each of the phalanges moving to a full range of motion toward the palm.

The prosthetic finger 100 may be controlled in a variety of ways. In some cases, the prosthetic finger 100 can be controlled using myoelectric sensors that are attached to a limb or other portion of a user. Signals from the myoelectric sensors may be used to determine an intended movement for the finger and control signals for controlling motion of the prosthetic finger may be generated using the signals from the myoelectric sensors.

In some cases, a control system, power source, and/or other components of the prosthetic finger 100 maybe contained in the socket or a separate housing that is worn or otherwise attached to the user. In other cases, the control system, power source, sensors, or other components can be integrated into the prosthetic finger 100. For example, one or more sensors can be internally or externally located on the prosthetic finger 100 and measure forces, movement, strain, or any other suitable parameters. In some cases, a control system, wired or wireless communications systems can be used to transmit control signals and/or other parameters to and from the prosthetic finger 100 and may be located on the prosthetic finger, for example, within a phalange of the prosthetic finger 100.

In some cases, a user may have multiple prosthetic fingers 100 attached to their hand. Each prosthetic finger may be independently controlled, for example, based on outputs from a myoelectric sensing system. Accordingly, in cases where a user has multiple prosthetic fingers 100, each prosthetic finger 100 may move independently of other prosthetic fingers 100.

FIG. 1B show an example of a prosthetic hand 105 that includes multiple powered prosthetic fingers 100. In cases where a user has a partial or complete hand amputation, the user may have a partial or complete prosthetic hand 105 (also referred to herein as a “hand”). The prosthetic hand 105 may include one or more prosthetic fingers 100 as well as other components such as a sensing system, control system, power source, and so on. Accordingly, the prosthetic fingers described herein may be attached to a user using a passive prosthetic socket or form a part of a prosthetic hand 105 that includes one or more prosthetic fingers 100 and/or other components such as a sensing system, power source, control system and so on integrated together.

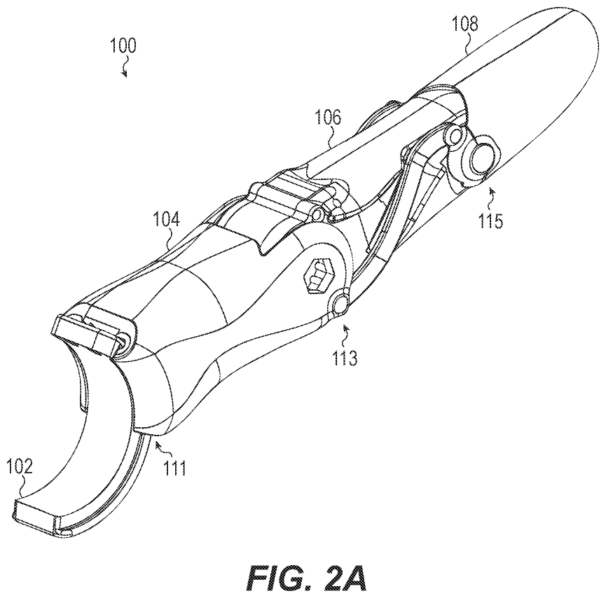

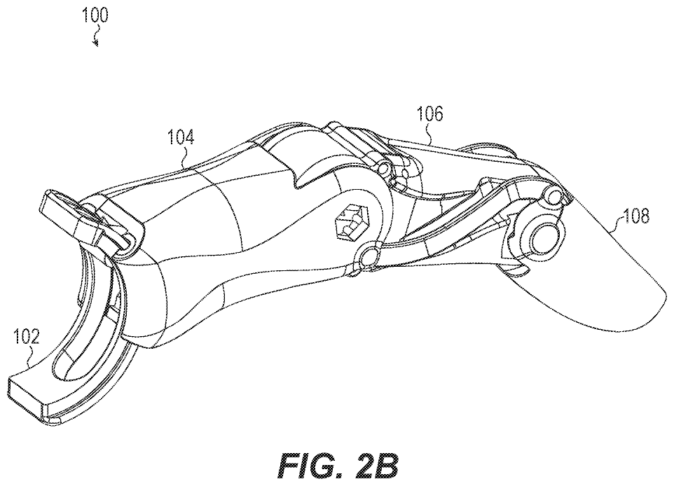

FIG. 2A shows an example of the powered prosthetic finger 100. The prosthetic finger 100 can include a track 102, a first phalange 104, a second phalange 106, and a third phalange 108. The track 102 and the first phalange 104 can be coupled to define a first joint 111 having a first movement profile. In three-phalange systems, the first joint 111 may correspond to an MCP joint and the track 102 and the first phalange 104 may be configured such that the first movement profile emulates movement of an MCP joint in a natural finger. The first phalange 104 and the second phalange 106 can be coupled to define a second joint 113 having a second movement profile. In three phalange systems, the second joint 113 may correspond to a PIP joint of a natural finger. The second phalange 106 and the third phalange 108 can be coupled to define a third joint 115 having a third movement profile which, in three phalange systems, may correspond to a DIP joint of a natural finger.

In some cases, the track 102 can have a curved profile and the first phalange 104 can move along the curved profile of the track 102 to define the first movement profile. During a flexion movement, the curved profile of the track 102 causes a base of the first phalange 104 to move downward and toward the palm of a user, which causes a distal end of the first phalange 104 to rotate towards the palm of the user (e.g., shown in FIG. 2B). The curved profile of the track 102 may allow the prosthetic finger 100 to be externally mounted to a socket or other prosthesis, while emulating the internal motion of a natural MCP joint. For example, the curved profile of the track can define a center of rotation of the first phalange 104 that is external to the prosthetic finger 100 and located within the socket or hand portion of the user. Accordingly, the curved profile of the track 102 may help emulate the internal motion of the MCP joint in an externally mounted device.

In other cases, the track 102 and the first phalange 104 can be coupled to define other movement profiles. For example, the first phalange 104 can be rotationally coupled to the track 102, but may not move along the track 102 as described above. In these examples, the first phalange 104 may rotate about an axis defined by the coupling of the first phalange 104 to the track 102 as the prosthetic finger moves in flexion and extension. In these examples, the center of rotation may be externally located at the interface of the first phalange 104 and the track 102.

The first phalange 104 and the second phalange 106 can be rotationally coupled and the second movement profile includes rotation of the second phalange 106 with respect to the first phalange 104. The second joint 113 can be defined by the coupling of the distal end of the first phalange 104 to a proximal end of the second phalange 106. During flexion, the second phalange 106 can rotate about the first phalange 104 downward and toward the track 102 (shown in FIG. 2B). During extension, the second phalange 106 can rotate about the first phalange 104 upward and away from the track 102. The combined motion of the first joint 111 and the second joint 113 can define the second movement profile which can cause the base of the second phalange 106 to both move (translate) and rotate toward a palm of a user thereby emulating the grasping motion of a natural finger.

The second phalange 106 and the third phalange 108 can be rotationally coupled and the third movement profile includes rotation of the third phalange 108 with respect to the second phalange 106. The third joint can be defined by the coupling of the distal end of the second phalange 106 to the proximal end of the third phalange 108. During flexion, the third phalange 108 can rotate about the second phalange 106 downward and toward the track 102 (shown in FIG. 2B). During extension, the third phalange 108 can rotate about the second phalange 106 upward and away from the track 102. The combined motion of the first joint 111, the second joint 113, and the third joint 115 can define the third movement profile which can cause the base of the third phalange 108 to both move (translate) and rotate toward a palm of a user thereby emulating the grasping motion of a natural finger.

FIG. 2B shows an example of the prosthetic finger 100 in partial flexion. During flexion, the first phalange 104 may move along the track 102 causing the first, second, and third phalanges 104, 106, and 108 to move downward and toward a palm of a user. The second phalange 106 may additionally rotate with respect to the first phalange 104 and the third phalange 108 may rotate with respect to the second phalange 106 as described herein. Movement of the prosthetic finger 100 can be caused by a motor and gear system as described herein and the movement profile of the phalanges can be defined by a linkage system as described herein. The linkage system can cause the coordinated movement of each of the phalanges. For example, during extension or flexion, the linkage system can cause the movement of the first phalange 104 along the track 102, rotation of the second phalange 106 about the first phalange 104 and rotation of the third phalange 108 about the second phalange 106 to occur at the same time.

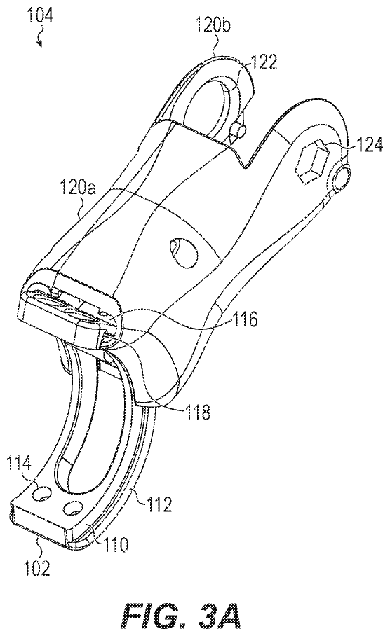

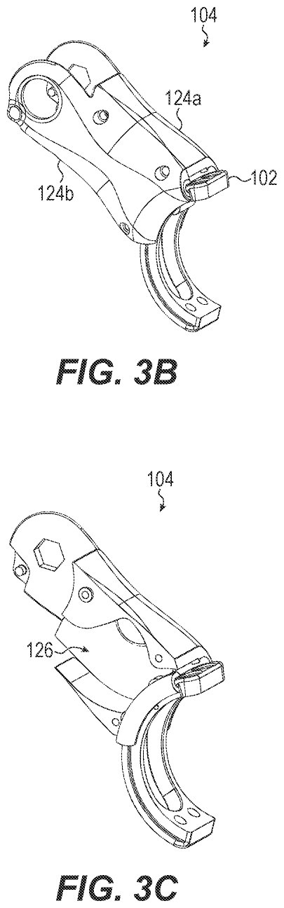

FIG. 3A shows an example of the first phalange 104 coupled to the track 102. The track 102 may define a central portion 110 and one or more rails 112 (only one of which is labeled for clarity) that extend from the body portion 110. The rails 112 can be used to couple the first phalange 104 to the track 102. For example, the first phalange 104 can include one or more slides 116 that partially surround the rails 112 and couple the first phalange 104 to the track 102 and allow the first phalange 104 to slide along the rails 112. In other cases, the rails 112 can be internal rails and the first phalange 104 can include features that insert into and slide along the internal rails. In some cases, the rails 112 and/or the slides 116 can include materials that facilitate sliding of the first phalange 104 along the track 102. For example, the rails 112 and/or the slides can include lower friction materials, wear resistance materials, coatings, bearings and/or materials with other suitable properties that facilitate the sliding interaction between the first phalange 104 and the track 102.

The track 102 can include stops that limit the range of travel of the first phalange 104 along the track 102 and/or prevent the first phalange 104 from disengaging with the rails 112. The track 102 may define a first stop 118 which can include a ledge or any other suitable feature that stops the first phalange 104 from sliding further along the track 102. In some cases, the first stop 118 may be located at an upper portion of the track 102 as shown. The first stop 118 can be located at a lower portion or at other locations along the track 102. A second stop (not shown) can be removably couple to the track 102, which can allow for the first phalange 104 to be coupled and decoupled from the track 102. For example, the second stop can be attached to the track 102 after the first phalange 104 has been engaged with the rails 112.

The track 102 can include features for attaching the prosthetic finger 100 to a hand of a user. In some cases, the track 102 can include one or more openings 114 that allow the track to be coupled to a prosthetic socket or other prosthetic using fasteners such as screws, bolts, pins, or any other suitable fastener. In other cases, the track 102 may include other fastening features such as a bar/rod that is captured by a hook or other feature on a prosthetic socket.

The first phalange 104 can include a body portion 120a and a distal portion 120b that couples to the second phalange 106. The distal portion 120b can define a first opening 122 that forms a rotational coupling with the second phalange 106. For example, a bearing, sleeve, washer or other component may be positioned within the first opening 122 and allow the first phalange 104 and the second phalange 106 to rotate with respect to each other. The distal portion 120b may also define a second opening 124, which may be a keyed opening or have other features that engage with a drive system output shaft. In some cases, bolting or pinning can be used to couple the drive system output shaft to the first phalange.

The second opening 124 may be configured to couple with an output from a gear system that is attached to the second phalange 106, as described herein. Accordingly, in response to the output from the gear system rotating, the keyed features of the second opening 124 can prevent rotation of the output with respect to the first phalange 104 thereby causing the first phalange 104, the second phalange 106 and the third phalange 108 to move as described herein. The second opening 124 can be configured in variety of ways including a keyed feature as shown in FIG. 3A. In other cases, the second opening 124 can be circular and/or press fit, welded, or otherwise fixed to an output component from the gear system.

FIG. 3B shows another perspective view of the track 102 and the first phalange 104 from the opposite side shown in FIG. 3A. In some cases, the first phalange 104 can be formed from multiple parts that are coupled together. For example, the first phalange 104 can include a first body portion 124a and a second body portion 124b that is coupled to the first body portion 124a. In other cases, the first phalange 104 can include three or more parts. For example, the first phalange 104 can include the first (side) body portion 124a, a second (middle) body portion, and a third (side) body portion that is opposite the first body portion.

FIG. 3C shows a perspective view of the track 102 and the first phalange 104 as shown in FIG. 3B, but with the second body portion 124b removed. The first phalange 104 can define an interior cavity 126. In some cases, a linkage system that couples movement of the track and phalanges can extend through the interior cavity 126 as described herein.

The track 102, the first phalange 104, the second phalange 106, and the third phalange 108 can be formed from a variety of materials including metals, metal-alloys, polymer materials, ceramics, or combinations thereof or other suitable materials.

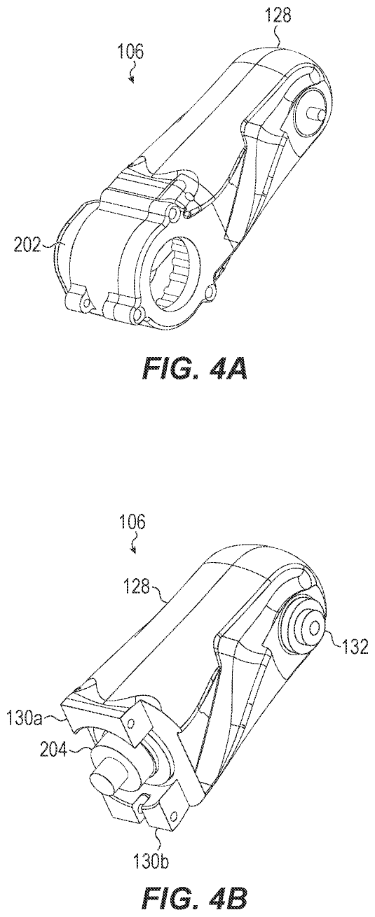

FIGS. 4A and 4B show examples of the second phalange 106 of the prosthetic finger 100 described herein. FIG. 4A shows an example of the second phalange 106 with a gear system 202 attached and FIG. 4B shows an example of the second phalange 106 with the gear system removed. The second phalange 106 can include a body portion 128, a motor 204 and a gear system 202. The motor 204 and the gear system 202 can be coupled to the body 128. The gear system 202 may define a proximal end of the second phalange 106 and interface with a distal portion (e.g., distal portion 120b) of the first phalange 104 to define the second joint. The body portion 128 may define a distal end of the second phalange 106 and interface with the third phalange 108 to define the third joint.

As shown in FIG. 4B, the motor 204 can be positioned within an interior of the second phalange 106. For example, a base portion of the motor 204 may be positioned at a distal end of the interior of the second phalange 106 and an output portion of the motor 204 may interface with the gear system 202. The body portion 128 can define mounting features 130a, 130b for coupling to the gear system 202. The body portion 128 may also define rotational features 132 which may be used to rotationally couple to the third phalange 108. For example, the rotational features 132 may be inserted into the opening on the third phalange 108. In some cases, bearings, bushings or other components may be used to couple the third phalange 108 to the rotational features 132 on the second phalange 106.

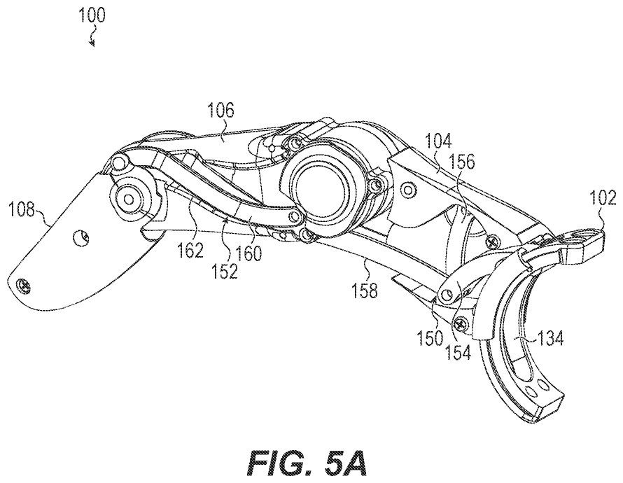

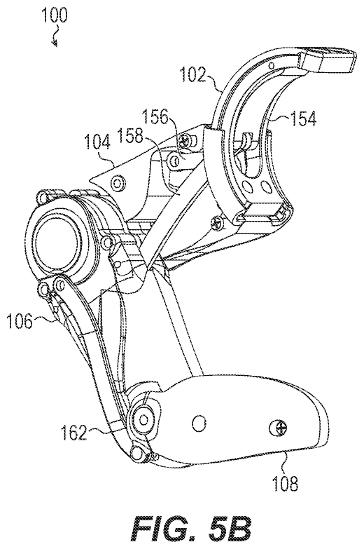

FIG. 5 shows an example of the linkage system for the prosthetic finger 100 described herein. The linkage system can include a first linkage set 150 that couples motion of the track 102, the first phalange 104 and the second phalange 106 and a second linkage set 152 which couples motion of the third phalange 108 to the track 102, the first phalange 104, and the second phalange 106.

The first linkage set 150 can include multiple link bars that are coupled together to define the movement profiles of the first phalange 104 and the second phalange 106. The first linkage set 150 can include a first link bar 154, a second link bar 156, and a third link bar 158. The first link bar 154 can have a first end that is coupled to the track 102 and a second end that is positioned inside the first phalange 104. The first end of the first link bar 154 can be rotationally coupled to the track, for example using a pin, and the first link bar 154 can rotate about the coupling.

The second link bar 156 can have a first end that is coupled to the first phalange 104 and a second end that is coupled to the second end of the first link bar 154. The first end of the second link bar 156 can be rotationally coupled to the first phalange 104 such that the second link bar 156 can rotate with respect to the first phalange 104.

The third link bar 158 can have a first end that is coupled to the second phalange 106 (e.g., a bottom mounting feature 130b of the second phalange 106 shown in FIG. 4B) and a second end that is coupled to the second ends of the first link bar 154 and the second link bar 156. The first end of the third link bar 158 may be rotationally coupled to the second phalange 106 such that the third link bar 158 can rotate with respect to the second phalange 106. The second ends of each of the first link bar 154, the second link bar 156, and the third link bar 158 can be rotationally coupled together such that each of these link bars can rotate with respect to each other.

The multi-link bar system of the first linkage set 150 can be configured to maintain the second ends of each link bar within the first phalange 104 as the prosthetic finger 100 moves in flexion and extension. For example, the second link bar 156 may define a pivot point between the first link bar 154 and the third link bar 158, that decreases the protrusion of the linkage set 150 from the interior of the first phalange 104 as compared to a single link bar system (e.g., where a single link bar couples the second phalange 106 to the track 102.

In some cases, the first end of the first link bar 154 can be positioned within an opening 134 defined by the track 102. As shown in FIG. 5B, as the prosthetic finger 100 moves in flexion, the first link bar 154 may rotate into the opening 134 in the track 102.

In some cases, one or more of the link bars in the first linkage set 150 can have a curved profile. For example, the first link bar 154 can have a curvature that matches the curvature of the track 102, which may allow the first link bar to nest within the opening 134 of the track 102 (e.g., as shown in FIG. 5B). The nesting of the first link bar 154 within the opening 134 of the track may reduce interference with other components and/or decrease the profile, size and/or mass of the prosthetic finger 100.

The second link bar 156 may have a curved profile, which can allow it to bend around the internal structure of the first phalange 104, for example, as shown in FIG. 5B. This may allow for the first phalange 104 to include thicker body sections that increase a strength, rigidity, or other mechanical properties while reducing interference between the first linkage set 150 and the first phalange 104 and/or increasing the range of motion of the prosthetic finger 100.

The third link bar 158 may have a curved profile, which may increase an amount of the third link bar 158 that is maintained within the interior of the first phalange 104 throughout the range of motion of the prosthetic finger 100, for example as compared to a straight link bar design. The curved profile of the third link bar 158 may reduce interface of the link bar with an object that is being grasped by the user.

The second linkage set 152 can include one or more link bars 160 (one of which is shown in FIG. 5A). In some cases, the second linkage set 152 can include a first link bar 160 positioned on a first side of the second phalange 106 and a second link bar (not shown in FIG. 5A) positioned on a second side of the second phalange 106. Each link bar 160 can have a first end coupled to the first phalange 104 and a second end coupled to the third phalange 108. The second linkage set 152 can cause the third phalange 108 to rotate about the second phalange 106 to move in flexion and extension as described herein. In some cases, the second phalange 106 can define one or more channels 162 and the link bar(s) 160 can be positioned within a channel 162.

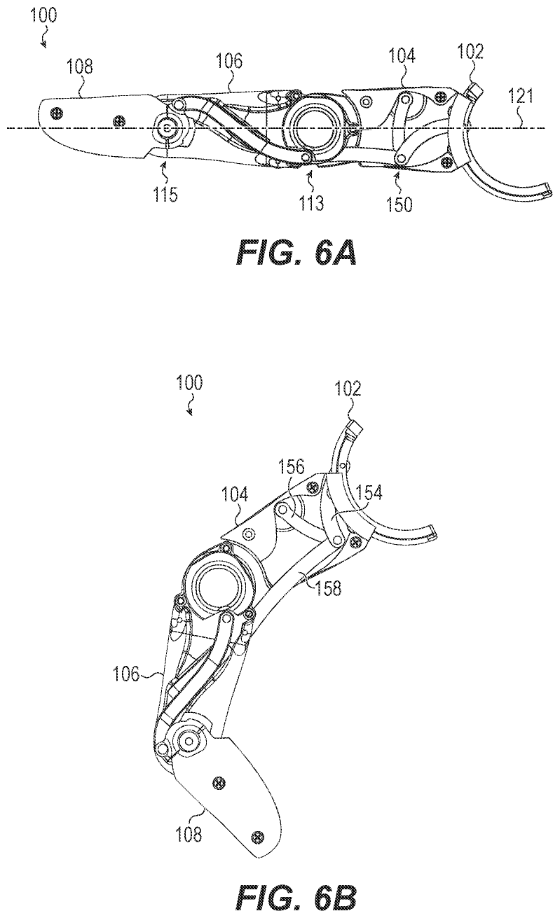

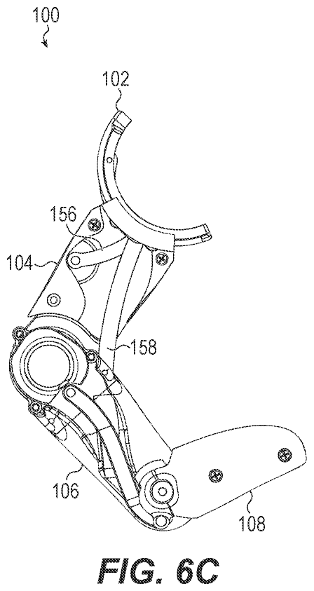

FIGS. 6A-6C show examples of movement of the prosthetic finger 100 from a full extension position to a full flexion position. FIG. 6A shows an example of the prosthetic finger 100 in a full extension position. In the full extension position a length of each of the first phalange 104, the second phalange 106 and the third phalange 108 may be substantially aligned. For example, a length of each of the first phalange 104, the second phalange 106, and the third phalange 108 may align along line 121. In other cases, at a full flexion position, the first phalange 104, the second phalange 106 and the third phalange 108 may not be aligned along a straight line (e.g., they may be slightly bent, have a curved profile, or the like). In some cases, in the full extension position, a rotational axis of the second joint 113 and the third joint 115 may be aligned along line 121, which extends substantially through a vertical mid-point of each of the first phalange 104, the second phalange 106, and the third phalange 108.

In the full flexion position, the first linkage set 150 may be positioned within the prosthetic finger 100 and substantially within the first phalange 104. As the prosthetic finger 100 moves in flexion, the first phalange 104 moves downward along the track 102, the second phalange 106 rotates about the first phalange 104 and the third phalange 108 rotates about the second phalange 106, as described herein. The coupled second ends of the first, second and third link bars 154, 156, 158 move towards the track and remain within the first phalange 104. FIG. 6B shows an intermediate position of the prosthetic finger 100. In the intermediate position, the majority of the first linkage set 150 remains within the first phalange 104 and only a portion of the third link bar 158 extending out of the prosthetic finger near the second joint 113. The curved profile of the third link bar 158 can reduce the amount of the link bar that extends from the bottom side of the prosthetic finger 100.

FIG. 6C shows the prosthetic finger 100 in a full flexion position. As the prosthetic finger moves into the full flection position, the first link bar 154 can nest within an opening in the track 102 as described herein. The second link bar 156 may remain within the first phalange 104 throughout the range of movement from full extension to full flexion and the majority of the third link bar 158 may remain within the first phalange 104. This movement profile defined by the first linkage set 150 may reduce interface of the linkage system into the grasping space (e.g., space below the base of the first phalange 104) as compared to other link systems such as a single link extending between the track 102 and the second phalange 106.

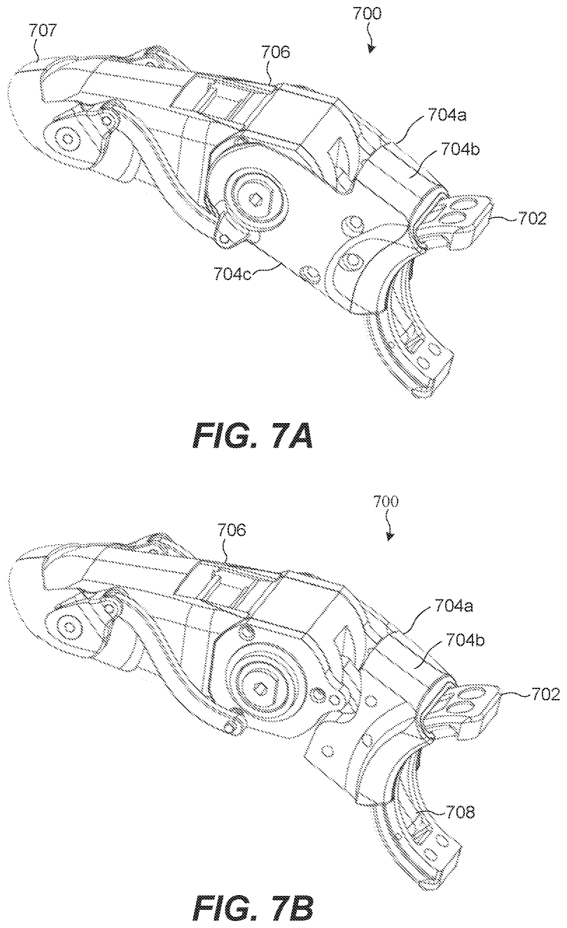

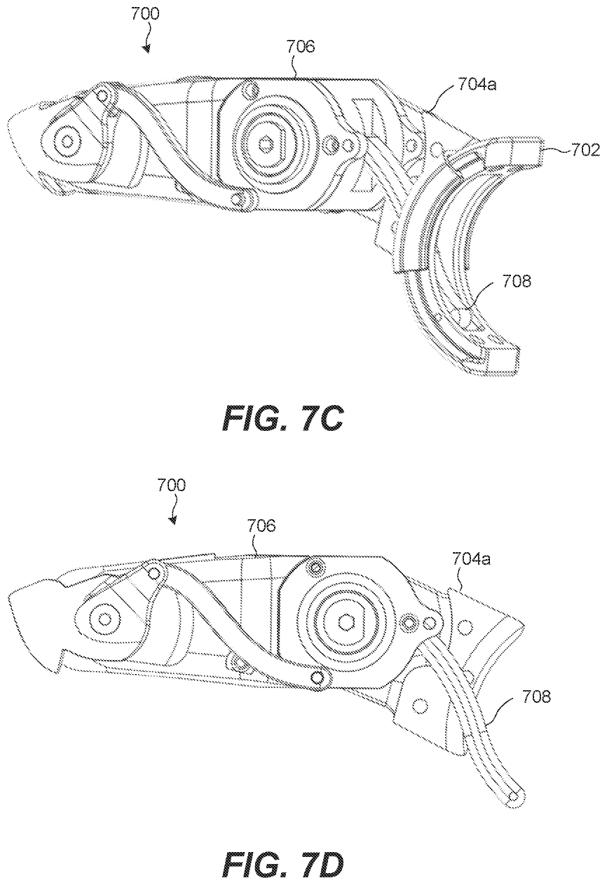

FIGS. 7A-7D show an example of a powered prosthetic finger 700 with a single link bar positioned at least partially in a first phalange. The powered prosthetic finger 700 can be an example of the prosthetic fingers described herein (e.g., prosthetic finger 100). The prosthetic finger 700 can include a track 702, a first phalange 704, a second phalange 706 and a third phalange 707. The first phalange 704 can have a three-part structure that includes a first side section 704a, a middle body section 704b and a second side section 704c. The multiple sections can be coupled together in any suitable manner including using threaded fasteners, pinning, adhesives, welding and/or the like. The three phalange sections 704 can define an internal space within the first phalange 704 as described herein.

As shown in FIG. 7B, the prosthetic finger 700 can include a link bar 708 that has a first end that coupled to the track 702 and a second end that couples to the second phalange 706 (e.g., a portion of the gear system as described herein). The single link bar 708 can extended through the interior space defined by the first phalange 704. The first phalange 704 can define an interior space that allows the link bar 708 to move within the first phalange 704 as the prosthetic finger 700 moves in extension and flexion.

As shown in FIG. 7C, the first end of the link bar 708 can be rotationally coupled to the track 702. The track 702 can define an opening that the link bar 708 can move (e.g., rotate) within as the prosthetic finger moves in flexion and tension. The second end of the link bar 708 can be rotationally coupled to the second phalange. The coupling of the link bar 708 can cause the second phalange to 706 to move in flexion as the first phalange 704 moves in a first direction along the track (e.g., from top to bottom of the track 702 as shown in the orientation of FIG. 7C). The link bar 708 can cause the second phalange 706 to move in extension as the first phalange 704 moves in a second direction along the track. As the first phalange 704 and the second phalange 706 move in flexion and extension, the link bar 708 may pivot into and out of the opening defined by the track 702 as described herein.

As shown in FIG. 7D, the link bar 708 can have a curved profile along its length, which may help maintain the link bar 708 within the internal space of the first phalange 704 as the prosthetic finger 700 moves in extension and flexion. For example, the curved profile of the link bar 708 may reduce (or eliminate) portions of the link bar 708 from entering the grasping space below the palmar surface of the first phalange 704.

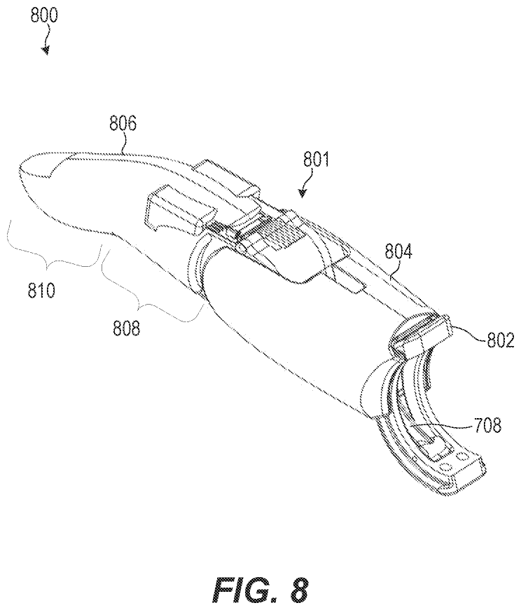

FIG. 8 shows an example of a powered prostatic finger 800 that has two phalange segments including a first phalange 804 coupled to a track 802, and a second phalange 806 rotatably coupled to the first phalange 804 at a joint 801. The track 802, the first phalange 804 and the second phalange 806 can be examples of the prosthetic fingers described herein. For example, the track 802 may be an example of the tracks 102 and/or 702; the first phalange may be an example of the first phalanges 104 and/or 704, and the second phalange 806 may be an example of the second phalanges 106 and/or 706.

The second phalange 806 can include a drive assembly which may have an output gear positioned at the first joint 801, as described herein. In the example shown in FIG. 8, the second phalange 806 may be distal most phalange of the powered prosthetic finger 800. The second phalange may have features that correspond to both a medial phalange and a distal phalange of a natural finger. For example, the second phalange 806 may define a first region 808 and a second region 810. In some cases, the first region 808 and the second region 810 may be formed from that same material(s). In other cases, the first region 808 and the second region 810 may be formed from different components and rigidly fixed together.

The second region 810 may be angled with respect to the first region 808. The angle of the second region 810 may function to help a user grasp objects with the tip of the prosthetic finger 800 and/or perform other functions that are performed by a distal phalange of a natural finger. Additionally or alternatively, the first region 808 and the second region 810 may have different materials. For example, the second region 810 may include a compliant material such as a polymer that aids gripping objects.

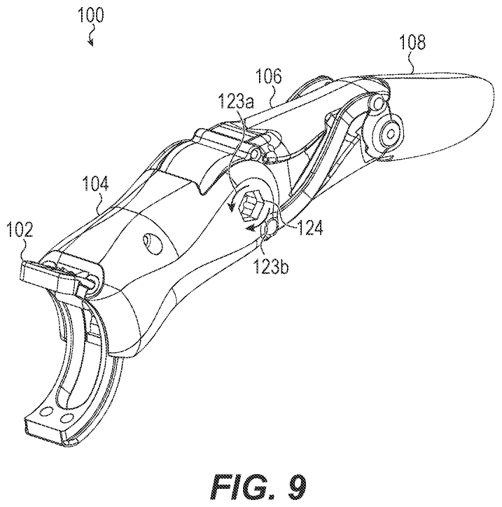

FIG. 9 shows an example of a drive system output on the prosthetic finger 100. The motor 204 (e.g., shown in FIGS. 4B, 9A and 9B) cause movement of the prosthetic finger 100 in flexion and extension by exerting an output force on the first phalange 104 at the second joint 113. For example, the motor 204 can be coupled to a gear system 202 as described herein and rotation of the motor can cause the gear system to output a torque on the first phalange 104 to cause the first phalange to rotate with respect to the second phalange. An output shaft of the gear system (not shown in FIG. 9) can extend into the keyed opening 124. The output shaft can have a keyed profile to prevent rotation of the shaft within the keyed opening 124. In some cases, the output shaft of the gear system can be coupled to the first phalange using bolts, pinning, welding, or any other suitable technique.

A motor output having a first direction can cause the output shaft of the gear system 202 to exert a first force on the first phalange 104 (e.g., a torque transmitted via the keyed opening 124) in a first direction 123a. The first force can cause the prosthetic finger 100 to move in flexion via the linkage system as described herein. A motor output having a second direction can cause the output shaft of the gear system 202 to exert a second force on the first phalange 104 (e.g., a torque transmitted via the keyed opening 124) in a second direction 123b. The second force can cause the prosthetic finger 100 to move in extension via the linkage system as described herein.

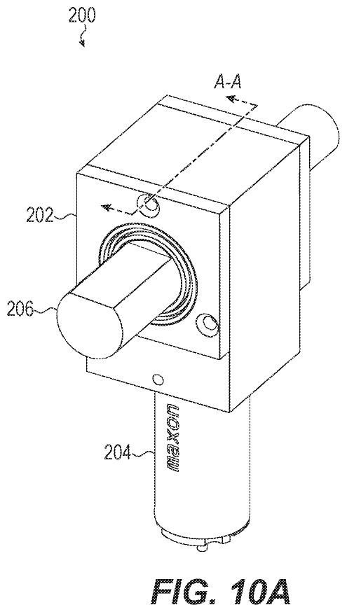

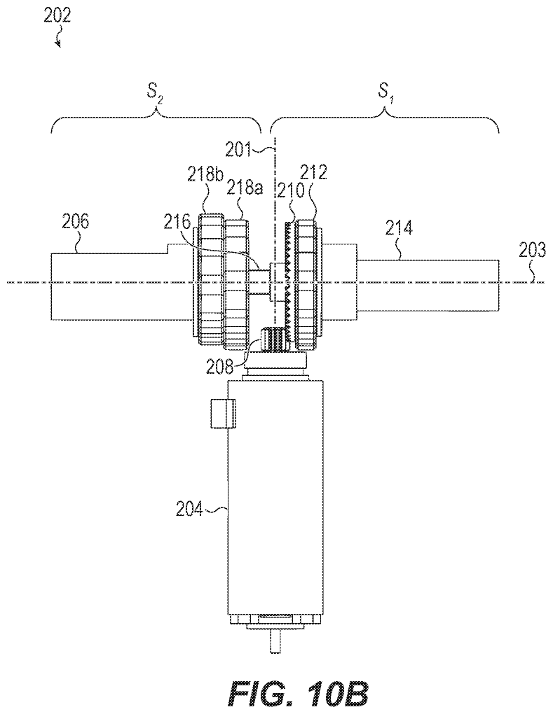

FIG. 10A shows an example of a drive system 200 of a powered prosthetic finger. The drive system 200 can include a motor 204 and a gear system 202, which may be integrated into the second phalange 106 as described herein. The motor 204 can be an electric motor that provides a rotational output to the gear system 202. The motor 204 can be any suitable electric motor include brushless, brushed, or stepper motors. The motor 204 can be controlled via one or more electrical inputs which may control a speed of rotation, direction of rotation, output force (e.g., torque), and so on.

The gear system 202 can be a cycloidal gear system that decreases the speed and increase the force of the motor output. The gear system 202 can include an input gear that interfaces with the motor 204 and an output gear that is coupled to the prosthetic finger 100 as described herein. The gear system can include an output shaft 206, which can couple to the first phalange (e.g., opening 124 shown in FIGS. 3A and 7) to cause movement of the prosthetic finger in response to a motor output.

FIG. 10B shows an example of the motor 204 and gear system 202 with the housing of the gear system 202 removed. The motor 204 can include a first gear 208 which transfers output motion of the motor 204 to the gear system 202. The first gear 208 can be a pinion gear (e.g., spur or helical gear) that meshes with an input gear 210 (e.g., face gear) of the gear system 202. In other cases, the first gear 208 can be a bevel gear that meshes with a bevel gear on the motor. Additionally or alternatively the first gear 208 can be configured as a ring gear that meshes with a face gear on the motor. In different embodiments the gear teeth on the first gear 208 and/or the input gear can be straight, spiral, hypoid, or other suitable configuration. The first gear 208 may rotate about a first axis 201 and the input gear 210 can rotate about a second axis 203. In some cases, the first axis 201 and the second axis 203 can be orthogonal. In other examples, the first gear 208 and the input gear 210 may be positioned at orientations that are non-orthogonal.

The gear system 202 can include a multi-stage cycloidal gear. The example embodiments of the gear system 202 presented herein are described with respect to a two-stage cycloidal gear. However, the principles described herein can apply to gear systems with fewer or additional stages and can be implemented in other embodiments.

The gear system 202 can include a first gear stage (S1), which includes the input gear 210, an eccentric shaft (e.g., eccentric shaft 220 shown in FIG. 10), a first cycloidal disk 212, and a first output shaft 214. The first output shaft 214 can coupled to an input shaft 216, which drives the second gear stage (S2). The input shaft 216 can include eccentric features (e.g., eccentric features 226 shown in FIG. 10). The second gear stage (S2) can include a second cycloidal disk 218a, a third cycloidal disk 218b, and the output shaft 206. The motor 204 can drive the first gear stage S1, which drives the second gear stage S2 which causes movement of the prosthetic finger 100, via the output shaft 206, as described herein.

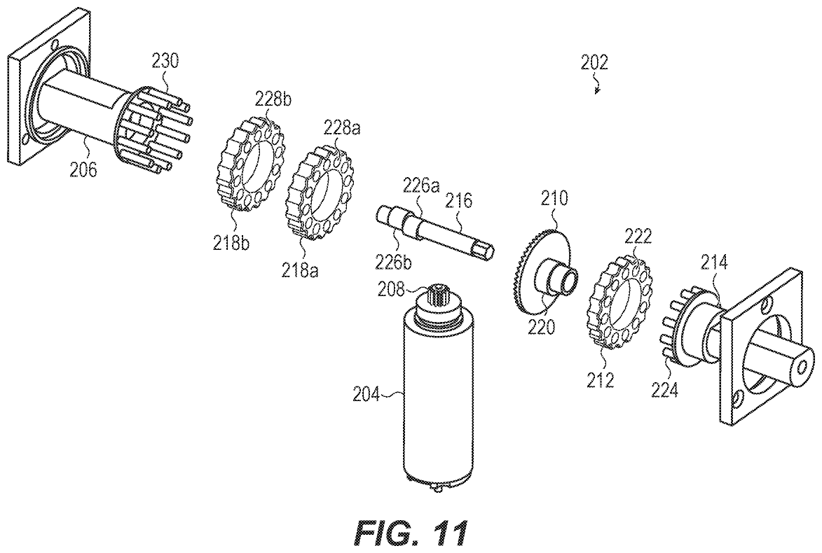

FIG. 11 shows an exploded view of the gear system 202 shown in FIG. 10 with part of the housing removed. The examples shown in FIGS. 9 and 10 also omit some of the gear system 202 components such as bearings, washers, fasteners, bushings and/or the like for the sake of illustration.

The eccentric shaft 220 can be coupled to the input gear 210 or formed as part of the input gear 210. The eccentric shaft 220 can be positioned off-center from the second axis 203 and rotation of the input gear 210 can cause the eccentric shaft 220 to rotate off-axis from the second axis 203. The first cycloidal disk 212 can be positioned over the eccentric shaft 220 and rotation of the input gear 210 can cause the cycloidal disk 212 to have an off-axis rotation profile defined by the eccentric shaft 220. In some cases, a rigid bearing can be placed over the eccentric shaft 220 and the first cycloidal disk 212 can be placed over the bearing. Accordingly, the input gear 210 and the eccentric shaft 220 can rotate at a first speed and the cycloidal disk 212 can rotate at a different speed.

The cycloidal disk 212 can define openings 222 (one of which is labeled for clarity) and the first output shaft 214 can include rollers 224 (one of which is labeled for clarity). Each roller 224 can be positioned within an opening 222. The cycloidal disk can also define a set of lobes along the outer profile. The housing (e.g., shown as housing 240 in FIG. 11) can include a complementary set of lobes that interface with the lobes on the first cycloidal disk 212. As the input gear 210 rotates, the eccentric shaft 220 causes the first cycloidal disk 212 to move in a cycloidal profile defined by the contact of the lobes on the cycloidal gear with the lobes on the housing, which, drives the rollers 224 and causes the first output shaft 214 to rotate about the second axis 203 at a reduced rate from the motor input. In some cases, the first gear stage can include multiple cycloidal disks, which may include two cycloidal disks that each eccentrically rotate out of phase from each other. In other cases, the rollers 224 can be positioned on the cycloidal disk 212 and the openings 222 can be positioned on the first output shaft.

The first output shaft 214 can be coupled to the input shaft 216 and rotation of the first output shaft 214 can cause the input shaft 216 to rotate. The input shaft 216 can define eccentric features 226, which can drive the second and third cycloidal disks 218. The second and third cycloidal disks 218 can each define openings 228. The output shaft 206 can include rollers 230 (which may also be referred to as “carrier pins”) that are positioned within the openings 228. Rotation of the input shaft 216 can drive the eccentric rotation of cycloidal disks 218, which cause the output shaft 206 to rotate about the second axis 203. In other cases, the second gear stage can include a single cycloidal disk 218 that includes openings 228. In other embodiments of the single cycloidal disk 218, the rollers 230 can be located on the cycloidal disk 218 and the openings can be located on the output shaft 206.

The second gear stage can include two cycloidal disks 218a and 218b, which can move out-of-phase with each other as defined by the eccentric features 226. For example, a first eccentric feature 226a can be configured to be 180 degrees out of phase with a second eccentric feature 226b, which can cause the first cycloidal disk 218a to rotate at 180 degrees out of phase from the second cycloidal disk 218b. The phase difference can help reduce vibration and/or other forces on the gear system 202. Additionally or alternatively, the first gear stage can include two cycloid disks that are configured to move out-of-phase. In some cases, the mass balancing can be used to reduce vibration and/or other forces (e.g., radial forces) on the assembly. For example, the input gear 210 can include an eccentric mass (e.g., machined or otherwise formed in the part, or added to the part) that is 180 degrees out of phase with the eccentric shaft 220.

The reduction ratio from the input gear 208 to the output shaft 206 can be set by modifying various aspects of the gear system 202 such as the input gear 208 and the first gear ratio, the design of the first stage (S1) cycloidal gear, the design of the second stage cycloidal gear (S2), combination thereof, or modifying other suitable parameters. In some cases, the total reduction ratio can be set to over 1000:1, over 1400:1, over 1600:1 or higher. In other cases, the reduction ratio can be set lower.

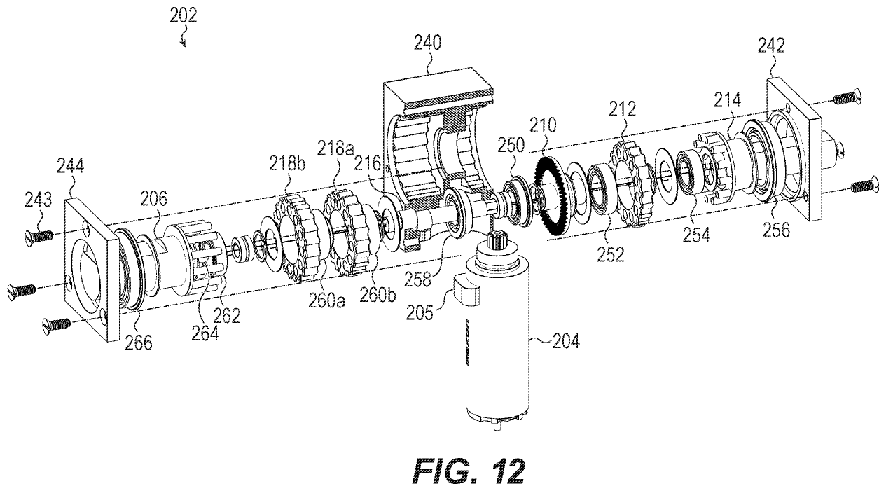

FIG. 12 shows an exploded view of components of the gear system 202. FIG. 12 shows an example of additional gear system components 202 including housing structures, bearings and other hardware components.

The gear system 202 can include a housing 240 that contains components of the gear system described herein. The housing 240 may define an opening for receiving the motor 204, a first set of internal features, such as first lobes, that form part of the first stage gear (S1) and a second set of internal features, such as second lobes, that form part of the second stage gear (S2). A first end cap 242 can couple to the housing 240 and contain components of the first gear stage (S1) and the second end cap 244 can couple to the housing 240 and contain components of the second gear stage (S2). In some cases, threaded fasteners 243 (one of which is labeled for clarity) can be used to couple the first end cap 242 and/or the second end cap 244 to the housing 240. Although in other embodiments, any other suitable fasteners and/or fastening methods can be used.

The motor 204 can be positioned at least partially within the opening defined by the housing 240. The motor 204 can be coupled to the housing 240 in a variety of ways. In some cases, a locking insert 205 can be used to fix the motor 204 with respect to the housing. For example, a screw can be used to push the locking insert 205 against the motor 204 housing thereby compressing the motor against the side of the housing 240. In other cases, the motor 204 can have keyed features which interface with keyed features on the housing 240 and prevent movement of the motor with respect to the housing. In further examples, the motor 204 can have threaded features that engage with threaded features in the housing 240, the motor 204 can be press-fit into the housing 240, the motor 204 can be welded or adhesively fastened, and/or attached to the housing 240 using any other suitable techniques.

In some cases, the second phalange 106 can be used to fix the motor with respect to the housing. For example, the second phalange 106 may couple to the housing 240 as described herein. In addition to or as an alternative to coupling the motor to the housing 240, the motor 204 can be coupled to the second phalange, using any suitable technique, and the coupling of the second phalange 106 to the housing 240 can fix the motor 204 with respect to the housing 240.

The first gear stage (S1) can include a first bearing 250, a second bearing 252, a third bearing 254 and a fourth bearing 256. The first gear stage (S1) can experience higher rotational speeds and lower forces as compared to subsequent gear stages such as the second gear stage (S2). In some cases, the first gear stage (S1) may include bearings or other components that are configured for higher rotational speeds and/or lower forces. For example, the first bearing 250, the second bearing 252, the third bearing 254, and the fourth bearing 256 can be ball bearings, roller bearings, and/or the like that perform well at higher rotational speeds. In other cases, the first gear stage bearings can include other rotational components such as bushings/sleeve bearings, and/or the like.

The second gear stage (S2) can include a fifth bearing 258, a sixth bearing 260a, a seventh bearing 260b, an eighth bearing 262, a spherical spacer 264, and a ninth bearing 266. The second gear stage (S2) can experience reduced rotational speeds and higher forces as compared to the first gear stage (S1). In some cases, the second gear stage (S2) may include bearings or other components that are configured for lower rotational speeds and/or higher forces. For example, various ones of the fifth bearing 258, the sixth bearing 260a, the seventh bearing 260b, the eighth bearing 262, and the ninth bearing 266 can include bushings/sleeve bearings. In other cases, the second gear stage bearings can include ball bearings, roller bearings, and/or the like.

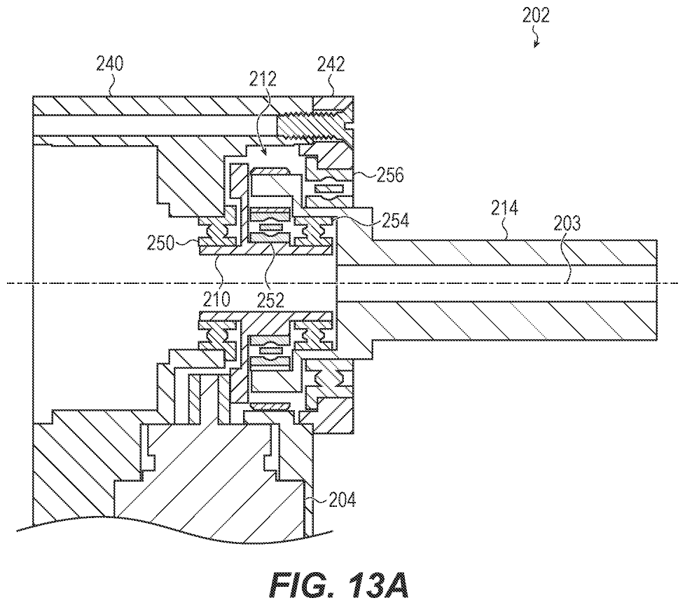

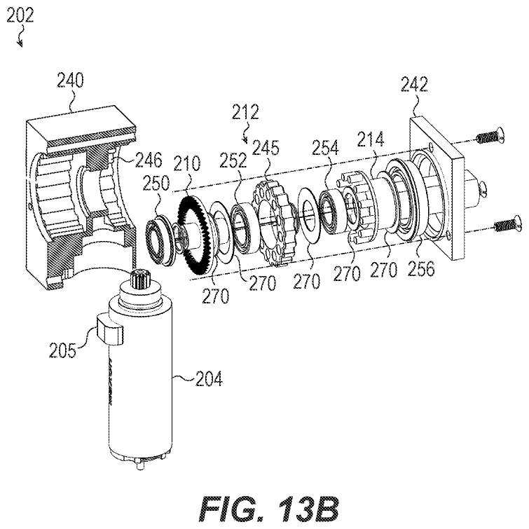

FIG. 13A shows a section view, taken along line A-A of FIG. 10A, of the first gear stage (S1) of the gear system 202. The first bearing 250 can be positioned between the housing 240 and the first gear 210 and allows the first gear to rotate with respect to the housing 240. The first gear 210 can include a first sleeve portion that extends along the second axis 203 and is positioned within the first bearing 250. The second bearing 252 can be positioned between the eccentric input shaft 220 and the first cycloidal disk 212, which provides a rigid support for the first cycloidal disk 212, while allowing the first cycloidal disk 212 and the eccentric input shaft 220 to rotate independently.

The third bearing 254 can be positioned between the first output shaft 214 and the first gear 210. The first gear 210 can include a second sleeve portion that extends along the second axis 203 and is positioned within the third bearing 254. The eccentric input shaft 220 can be formed on the first sleeve portion of the first gear 210 (e.g., the eccentric input shaft 220 and the first gear 210 are a continuous piece of material) or positioned over the first sleeve portion of the first gear 210 (e.g., the eccentric input shaft 220 is press fit over the sleeve of the first gear 210). The third bearing 254 can be positioned adjacent to the eccentric input shaft 220 and be concentrically positioned along the second axis 203 such that the first output shaft 214 rotates concentrically about the second axis 203. A fourth bearing 256 can be positioned between the first output shaft 214 and the first end cap 242. The fourth bearing 256 and the first output shaft 214 can be configured so that the first output shaft 214 rotates concentrically about the second axis 203.

The first output shaft 214 may extend through the first end cap 242. The first output shaft may be coupled with a bushing, bearing, and/or other component that is coupled to the first end cap 242. In other cases, the first output shaft 214 can terminate at/within the first end cap 242. For example, the first end cap 242 may define a solid outer surface and the first output shaft 214 is supported by the fourth bearing 256 within the first end cap 242, but does not extend out of the first end cap 242.

The bearings of the first gear stage (S1) can isolate the input shaft 216 (shown in FIGS. 10 and 11) from forces generated the first gear stage (S1) cycloidal assembly. For example, the bearings can couple (e.g., ground) the input gear 210 to the housing 240, which can prevent contact between the input gear and/or other components of the first cycloidal gear and the input shaft 216. This can effectively decouple the first gear stage (S1) components from forces (e.g.,) torques on the second gear stage (S2), which may result from external loading of the finger. Additionally or alternatively, the bearings can reduce and/or substantially eliminate radial loads transferred between the first gear stage (S1) and the second gear stage (S2). from the motion eccentric motion of the cycloidal disk 212 and to the input shaft 216, which can reduce the transfer of undesirable loads between the first gear stage (S1) and the second gear stage (S2). Accordingly, the gear system may be more robust to variations in alignment and/or other forces that occur from vibrations or other loads on the prosthetic finger.

FIG. 13B shows an exploded view of the first gear stage (S1) of the gear system 202 shown in FIG. 13A. The first cycloidal disk 212 can define a first set of lobes 245 along an outer circumference of the first cycloidal disk. The housing 240 can define a second set of lobes 246. The number of lobes in the first set of lobes 245 can be less (e.g., one less) than the number of lobes in the second set of lobes 246. The motion of the eccentric shaft 220 can drive the first set of lobes 245 along the second set of lobes 246, which causes rotation of the cycloidal disk 212 with respect to the housing 240. The rotation of the cycloidal disk 212 can drive the first output shaft 214, via the engagement of the rollers 224 (shown in FIG. 11) within the openings 222 (shown in FIG. 11).

In some cases, the gear system 202 can include shims 270, which can be used to define the axial positioning of one or more of the first gear stage (S1) components. Each of the shims 270 can be independently configured, including having different materials, thicknesses, inner and/or outer diameters, and so on. In some cases, the shims 270 can include thrust washers or other suitable types of bearing components.

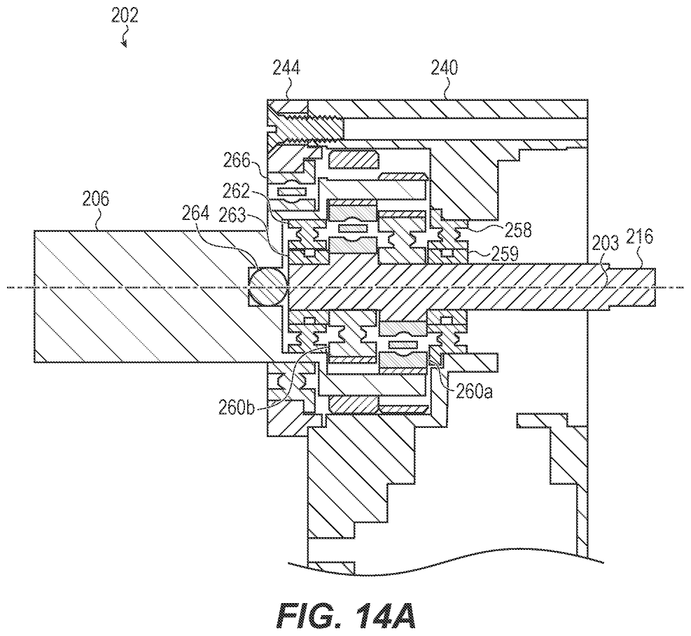

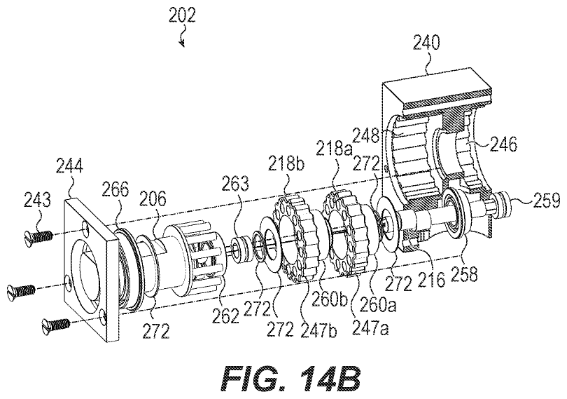

FIG. 14A shows a section view, taken along line A-A of FIG. 10A, of the second gear stage (S2) of the gear system 202. The fifth bearing 258 is positioned between the housing 240 and the input shaft 216. In some cases, a bearing sleeve 259 can be positioned over the input shaft 216 and between the fifth bearing 258 and the input shaft 216.

A sixth bearing 260a can be positioned between the first cycloidal disk 218a and the input shaft 216 and be positioned on the first eccentric feature 226a (shown in FIG. 11). A seventh bearing 260b can be positioned between the second cycloidal disk 218b and the input shaft 216 and be positioned on the second eccentric feature 226b (shown in FIG. 11). An eighth bearing 262 can be positioned between the output shaft 206 and the input shaft 216. The eighth bearing 262 can be positioned on an end portion of the input shaft 216 that is concentric to the second axis 203. A bearing sleeve 263 can be positioned over the input shaft 216 and between the eighth bearing 262 and the input shaft 216. In some cases, a spacer 264 can be positioned between the end of the input shaft 216 and the output gear 206. The spacer 264 can be a spherical spacers and may reduce friction, which may be caused by the differential rotational speeds of the input shaft 216 and the output gear 206. A ninth bearing 266 can be positioned between the output shaft 206 and the second end cap 244. The ninth bearing 266 and the output shaft 206 can be configured so that the output shaft 206 rotates concentrically about the second axis 203.

FIG. 14B shows an exploded view of the second gear stage (S2) of the example gear system 202 shown in FIG. 14A. The second cycloidal disk 218a can define a third set of lobes 247a along an outer circumference and the third cycloidal disk 218b can define a fourth set of lobes 247b along an outer circumference. The housing 240 can define a fifth set of lobes 248. The number of lobes in each of the third set of lobes 247a and the fourth set of lobes 247b can be less (e.g., one less) than the number of lobes in the fifth set of lobes 248. The motion of the eccentric portions of the input shaft 216 can drive the third set of lobes 247a and the fourth set of lobes 247b along the fifth set of lobes 248, which causes rotation of the cycloidal disks 218 with respect to the housing 240. The rotation of the cycloidal disk 218 can drive the output shaft 206, via the engagement of the rollers 230 (shown in FIG. 11) within the openings 228 (shown in FIG. 11).

In some cases, the gear system 202 can include shims 272, which can be used to define the axial positioning of one or more of the first gear stage (S1) components. Each of the shims 272 can be independently configured, including having different materials, thicknesses, inner and/or outer diameters, and so on. In some cases, the shims 272 can include thrust washers or other suitable types of bearing components.

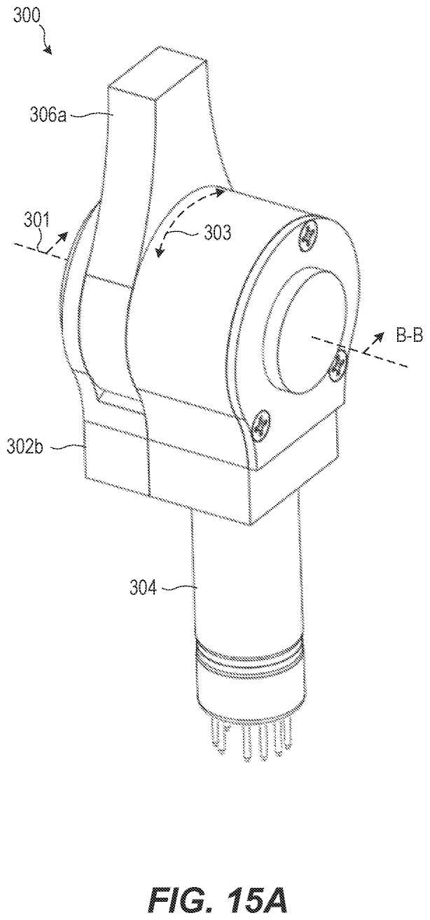

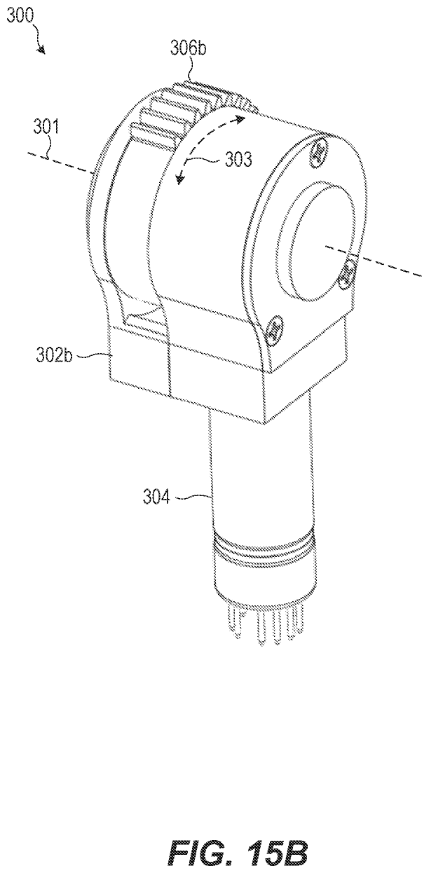

FIGS. 15A and 15B shows an example drive system 300 for a powered prosthetic finger. The drive system 300 can include a motor 304 and a gear system 302, which may be integrated into the second phalange 106 as described herein. The example drive system 300 can be an example of the drive systems described herein (e.g., drive system 200) and include a cycloidal gear system that drives movement of a prosthetic finger, such as the prosthetic fingers described herein. The gear system 302 can be a cycloidal gear system that decreases the speed and increase the force of the motor output.

The motor 304 can be an electric motor that provides a rotational output to the gear system 302. The motor 304 can be any suitable electric motor include brushless, brushed, or stepper motors. The motor 304 can be controlled via one or more electrical inputs which may control a speed of rotation, direction of rotation, output force (e.g., torque), and so on.

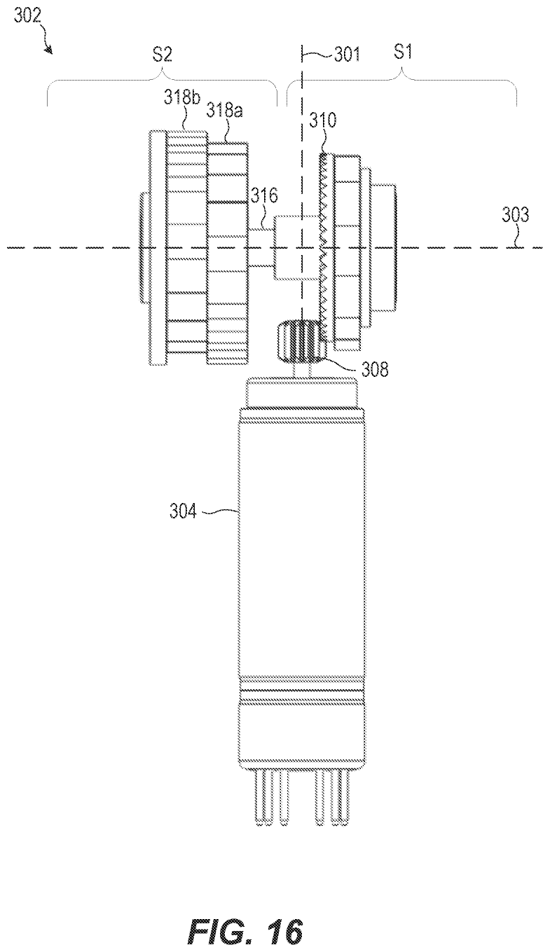

The gear system 302a shown in FIG. 15A can include an output flange 306a that is driven by the motor 304. The motor 304 can rotate an input gear (e.g., first gear 308 shown in FIG. 16) which can drive the cycloidal gear system 302a and cause the output flange 306a to rotate about an axis 301 in either direction along path 303. The output flange 306a can be coupled to the prosthetic finger 100, the prosthetic finger 700 or other prosthetic fingers, as described herein. The output flange 306a can cause movement of the prosthetic finger in response to a motor output. For example, the output flange 306a may couple to linkage system of a prosthetic finger, and can cause movement (e.g., flexion and extension of a prosthetic finger by rotating about axis 301 and moving in either direction along path 303).

The gear system 302b shown in FIG. 15B can include an output gear 306b that is driven by motor 304. The motor 304 can rotate an input gear (e.g., first gear 308 shown in FIG. 16) which can drive the cycloidal gear system 302b and cause the output gear 306b to rotate about an axis 301 gear teeth to move in either direction along path 303. The output gear 306b can be coupled to the prosthetic finger 100, the prosthetic finger 700 or other prosthetic fingers, as described herein. The output gear 306b can cause movement of the prosthetic finger in response to a motor output. For example, the output gear 306b may couple to linkage system of a prosthetic finger, and can cause movement (e.g., flexion and extension of a prosthetic finger by rotating about axis 301 and moving in either direction along path 303).

The drive system 300 may be coupled to a second phalange as described herein. For example, the motor 304 may be positioned within the second phalange and the gear system 302 may be located at a joint interface between a first phalange and the second phalange as described herein.

The drive system 200 described with reference to FIGS. 9-14B includes an annular cycloidal gear (e.g., the second set of lobes 248) that is grounded to the housing 240 thereby causing the output shaft 206 (and carrier pins/rollers 230) to rotate with respect to the housing 240 in response to the motor driving the gear system 202. The drive system 300 described with reference to FIGS. 15-19 includes carrier pins (e.g., pins 330) that are grounded to the housing 340 thereby causing an annular cycloidal gear (e.g., output flange 306a or output gear 306b) to rotate with respect to the housing.

FIG. 16 shows an example of the motor 304 and gear system 302 with the housing of the gear system 302 removed. The motor 304 can include a first gear 308 which transfers output motion of the motor 304 to the gear system 302. The first gear 308 can be a pinion gear (e.g., spur or helical gear) that meshes with an input gear 310 (e.g., face gear) of the gear system 302. In other cases, the first gear 308 can be a bevel gear that meshes with a bevel gear on the motor. Additionally or alternatively the first gear 308 can be configured as a ring gear that meshes with a face gear on the motor. In different embodiments the gear teeth on the first gear 308 and/or the input gear can be straight, spiral, hypoid, or other suitable configuration. The first gear 308 may rotate about a first axis 301 and the input gear 310 can rotate about a second axis 303. In some cases, the first axis 301 and the second axis 303 can be orthogonal. In other examples, the first gear 308 and the input gear 310 may be positioned at orientations that are non-orthogonal.

The gear system 302 can include a multi-stage cycloidal gear. The example embodiments of the gear system 302 presented herein are described with respect to a two-stage cycloidal gear. However, the principles described herein can apply to gear systems with fewer or additional stages and can be implemented in other embodiments.

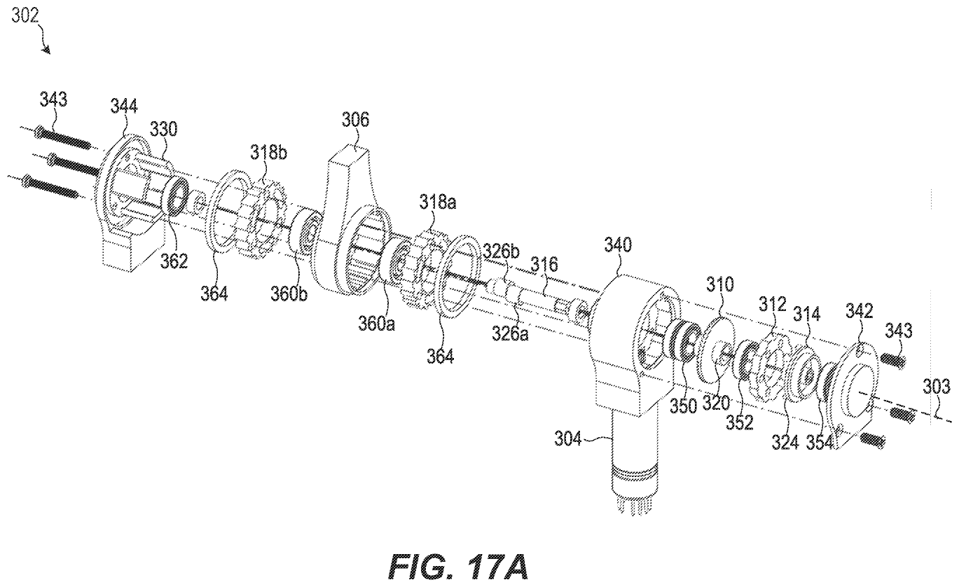

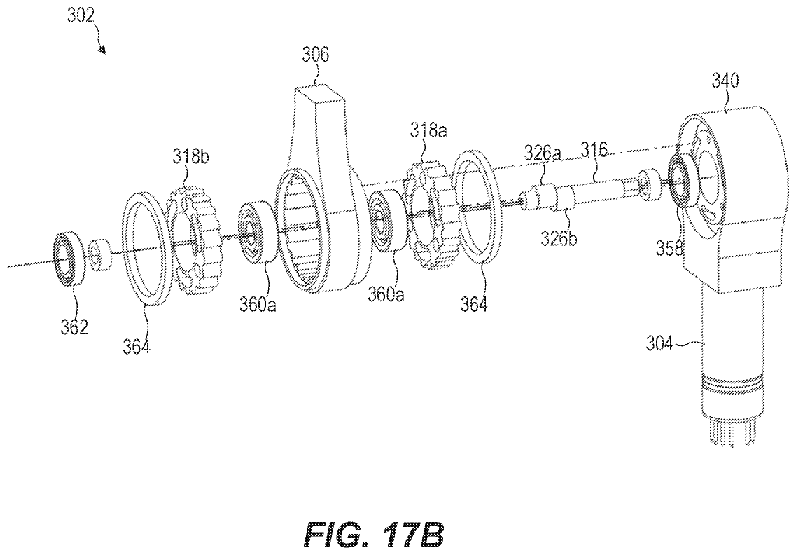

The gear system 302 can include a first gear stage (S1), which includes the input gear 310, an eccentric shaft (e.g., eccentric shaft 320 shown in FIG. 17A), and a first cycloidal disk 312. The first cycloidal disk 312 can coupled to an input shaft 316, which drives the second gear stage (S2). The input shaft 316 can include eccentric features (e.g., eccentric features 326 shown in FIG. 17A). The second gear stage (S2) can include a second cycloidal disk 318a and a third cycloidal disk 318b. which drive motion of the output flange 306a or the output gear 306b. The motor 204 can drive the first gear stage S1, which drives the second gear stage S2 which causes movement of the prosthetic finger 100, via the output flange or output flange 306a or the output gear 306b, as described herein.

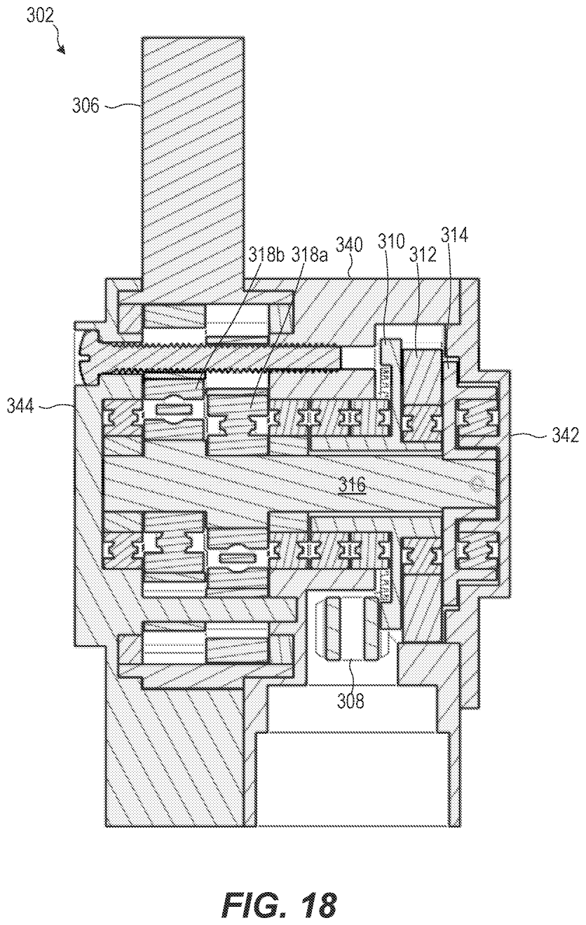

FIG. 17A shows a first exploded view of components of the gear system 302. FIG. 17A shows an example of additional gear system components 302 including housing structures, bearings and other hardware components. FIG. 17B shows a second exploded view of a portion of the gear system 302 shown in FIG. 17A. FIG. 18 shows a cross-sectional view of the drive system shown FIG. 15A taken along section B-B of FIG. 15A.

The gear system 302 can include a housing 340 that contains components of the gear system described herein. The housing 340 may define an opening for receiving the motor 304, a first set of internal features, such as first lobes, that form part of the first stage gear (S1). A first end cap 342 can couple to the housing 340 and contain components of the first gear stage (S1) and the second end cap 344 can couple to the housing 340 and contain components of the second gear stage (S2). In some cases, threaded fasteners 343 (some of which is labeled for clarity) can be used to couple the first end cap 342 and/or the second end cap 344 to the housing 340. Although in other embodiments, any other suitable fasteners and/or fastening methods can be used.

The motor 304 can be positioned at least partially within the opening defined by the housing 340. The motor 304 can be coupled to the housing 340 in a variety of ways. In some cases, a locking insert 305 can be used to fix the motor 304 with respect to the housing. For example, a screw can be used to push the locking insert 305 against the motor 304 housing thereby compressing the motor against the side of the housing 340. In other cases, the motor 304 can have keyed features which interface with keyed features on the housing 340 and prevent movement of the motor with respect to the housing. In further examples, the motor 304 can have threaded features that engage with threaded features in the housing 340, the motor 304 can be press-fit into the housing 304, the motor 304 can be welded or adhesively fastened, and/or attached to the housing 340 using any other suitable techniques.

In some cases, the second phalange 106 can be used to fix the motor with respect to the housing. For example, the second phalange 106 may couple to the housing 340 as described herein. In addition to or as an alternative to coupling the motor to the housing 340, the motor 304 can be coupled to the second phalange, using any suitable technique, and the coupling of the second phalange 106 to the housing 340 can fix the motor 304 with respect to the housing 340.

The first gear stage (S1) can include one or more first bearings 350 (one of which is labeled for clarity), a second bearing 352, and a third bearing 354. The first gear stage (S1) can experience higher rotational speeds and lower forces as compared to subsequent gear stages such as the second gear stage (S2). In some cases, the first gear stage (S1) may include bearings or other components that are configured for higher rotational speeds and/or lower forces. For example, the one or more first bearings 350, the second bearing 352, the third bearing 354, and the fourth bearing 356 can be ball bearings, roller bearings, and/or the like that perform well at higher rotational speeds. In other cases, the first gear stage bearings can include other rotational components such as bushings/sleeve bearings, and/or the like.

The second gear stage (S2) can include a fourth bearing 358 (shown in FIG. 17B), a fifth bearing 360a, a sixth bearing 360b, a seventh bearing 362, and one or more bushings 364. The second gear stage (S2) can experience reduced rotational speeds and higher forces as compared to the first gear stage (S1). In some cases, the second gear stage (S2) may include bearings or other components that are configured for lower rotational speeds and/or higher forces. For example, various ones of the fourth bearing 358, the fifth bearing 360a, the sixth bearing 360b, and the seventh bearing 362 can include bushings/sleeve bearings. In other cases, the second gear stage bearings can include ball bearings, roller bearings, and/or the like.

The one or more first bearings 350 can be positioned between the housing 340 and the first gear 310 and allows the first gear to rotate with respect to the housing 340. The first gear 310 can include an eccentric input shaft 320 that extends along the second axis 303 and is positioned within the second bearing 252. The second bearing 252 can be positioned between the eccentric input shaft 320 and the first cycloidal disk 312, which provides a rigid support for the first cycloidal disk 312, while allowing the first cycloidal disk 312 and the eccentric input shaft 320 to rotate independently.

The third bearing 354 can be positioned between a first output component 314 and the first end cap 342. The first gear 310 can include a second sleeve portion that extends along the second axis 303 and is positioned within the one or more first bearings 350. The eccentric input shaft 320 can be formed on the first sleeve portion of the first gear 310 (e.g., the eccentric input shaft 320 and the first gear 310 are a continuous piece of material) or positioned over the first sleeve portion of the first gear 310 (e.g., the eccentric input shaft 320 is press fit over the sleeve of the first gear 310). The third bearing 354 can be positioned between the first output component 314 and the first end cap 342.

The bearings of the first gear stage (S1) can isolate the input shaft 316 from forces generated by the first gear stage (S1) cycloidal assembly. For example, the bearings can couple (e.g., ground) the input gear 310 to the housing 340, which can prevent contact between the input gear and/or other components of the first cycloidal gear and the input shaft 316. This can effectively decouple the first gear stage (S1) components from forces (e.g.,) torques on the second gear stage (S2), which may result from external loading of the finger. Additionally or alternatively, the bearings can reduce and/or substantially eliminate radial loads transferred between the first gear stage (S1) and the second gear stage (S2). from the motion eccentric motion of the cycloidal disk 312 and to the input shaft 316, which can reduce the transfer of undesirable loads between the first gear stage (S1) and the second gear stage (S2). Accordingly, the gear system may be more robust to variations in alignment and/or other forces that occur from vibrations or other loads on the prosthetic finger.

The first cycloidal disk 312 can define a first set of lobes along an outer circumference of the first cycloidal disk. The housing 240 can define a second set of lobes, as described herein. The number of lobes in the first set of lobes can be less (e.g., one less) than the number of lobes in the second set of lobes. The motion of the eccentric shaft 320 can drive the first set of lobes along the second set of lobes, which causes rotation of the cycloidal disk 312 with respect to the housing 340. The rotation of the cycloidal disk 312 can drive the first output component 314, via the engagement of the rollers 324 within the openings in the cycloidal disk 312, as described herein. The first output component 314 can be coupled to the input shaft 316 (e.g., rigidly coupled to the input shaft 316) and cause the input shaft to rotate in response to a motor being driven.

In some cases, the gear system 302 can include shims, which can be used to define the axial positioning of one or more of the first gear stage (S1) components. Each of the shims can be independently configured, including having different materials, thicknesses, inner and/or outer diameters, and so on. In some cases, the shims can include thrust washers or other suitable types of bearing components.

The fourth bearing 358 can be positioned between the housing 340 and the input shaft 316. In some cases, a bearing sleeve can be positioned over the input shaft 316 and between the fourth bearing 358 and the input shaft 316.

The fifth bearing 360a can be positioned between the second cycloidal disk 318a and the input shaft 316 and be positioned on the first eccentric feature 326a. A sixth bearing 360b can be positioned between the third cycloidal disk 318b and the input shaft 316 and be positioned on the second eccentric feature 326b. A seventh bearing 362 can be positioned between the output the input shaft 316 and the second end cap 344.

The second cycloidal disk 318a can define a third set of lobes along an outer circumference and the third cycloidal disk 318b can define a fourth set of lobes along an outer circumference. The housing 340 can define a fifth set of lobes. The number of lobes in each of the third set of lobes and the fourth set of lobes can be less (e.g., one less) than the number of lobes in the fifth set of lobes.

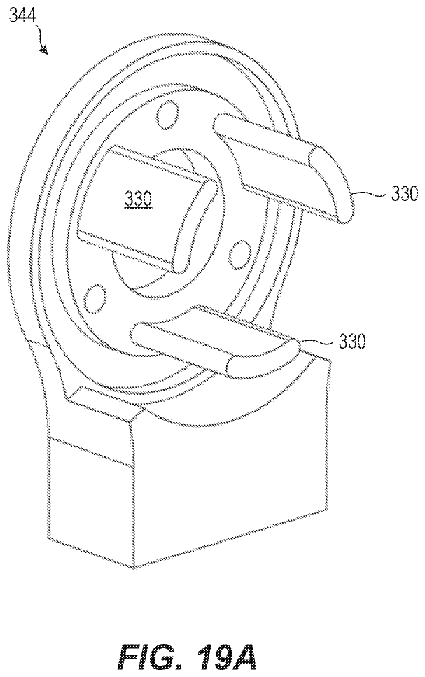

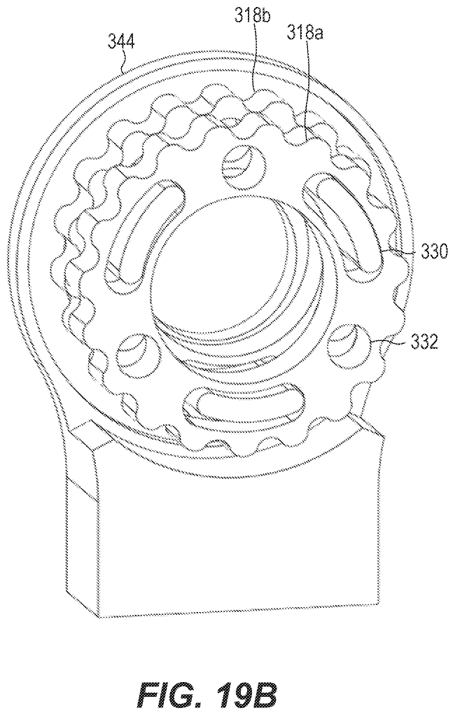

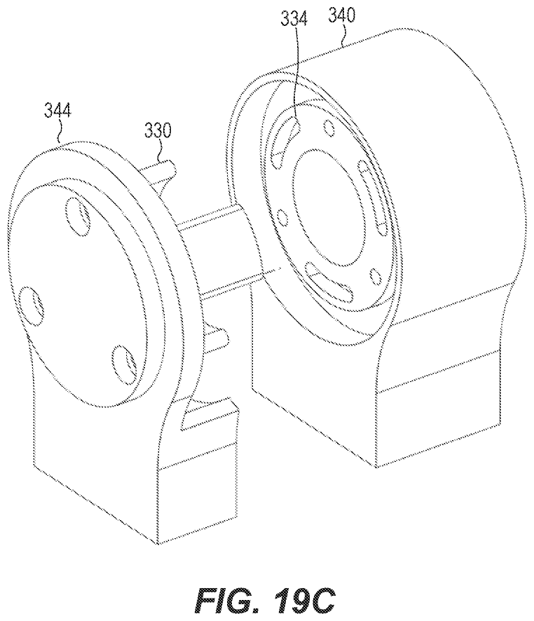

The second end cap 344 can define a set of carries pins 330 (one of which is labeled for clarity, and which may also be referred to herein as “lobes” or “rollers’). The carrier pins 330 may extend through openings in the second cycloid disk 318a and the third cycloidal disk 318b and couple to the housing 340. For example, the housing 340 may include openings (e.g., opening 334 shown in FIG. 19C) and the carrier pins 330 may extend into the openings on the housing 340. The carrier pins 330 maybe supported by the housing 340. In some cases, the carrier pins 330 may be press fit, or otherwise rigidly attached to the housing 340.

The carrier pins 330 and the openings in the cycloid disks can define the cycloidal movement of the cycloid disks 318, which can cause the output flange 306 to rotate concentrically around the axis 303. Accordingly, the motion of the eccentric portions of the input shaft 316 can drive the third set of lobes and the fourth set of lobes on the cycloid disks along the fifth set of lobes in the output flange 306, which causes movement of the cycloidal disks 318 with respect to the housing 340. The movement of the cycloidal disk 318 can drive the output flange 206c, output gear 306b, or other annular component, via the engagement of the lobes on the cycloidal disks 318 with the lobes on the output gear 306b.

FIG. 19A shows an example of the second end cap 344 for the drive system 300 described herein. The drive system 300 can include multiple carrier pins 330, which can define the cycloidal movement profile of the cycloidal disks, as described herein. The drive system 300 can have one or more carrier pins 330.

In some cases, the drive system 300 may increase a torque capacity of the system because each of the carrier pins 330 are support at each end. That is, the second end cap 344 supports each of the carrier pins 330 at one end and the housing 340 supports each of the carrier pins at the other end. In some cases, the carrier pins 330 can be a separate component from the second end cap 344 and press-fit or otherwise rigidly coupled to the second end cap 344. This may allow the carrier pins to be formed from materials with high resistance to wear.

FIG. 19B shows an example of the second end cap 344 with the second and third cycloidal disks 318 for the drive system 300 described herein. In some cases, the second cycloidal disk 318a and the third cycloidal disk 318b may be configured to eccentrically rotate out of phase from each other. For example, the second cycloidal disk 318a may be configured at 180 degrees out of phase with the third cycloidal disk 318b, which may reduce vibration of the system.

In some examples, the drive system 300 may include additional cycloidal disks on the S2 stage side and/or the S1 stage side. For example, in the S2 side may include three or more cycloidal disks. In these cases, each of the cycloidal disks may be configured to rotate at different phases, which may be spaced equally apart, which may reduce vibration of the system.

In some cases, the cycloidal disks (e.g., the first and second cycloidal disks 318) may include one or more second openings 332 and the fasteners (e.g., fasteners 343) may pass through the second openings 332 and couple the second end cap 344 to the housing 340. In some, cases the fasteners and openings 332 can be configured as cycloidal guides in addition to the carrier pins 330. For example, the fasteners may include a smooth section that extends through the openings 332 and the cycloidal disks 318 to guide the eccentric rotation of the cycloidal disks 318. In these example, the additional fasteners (functioning as additional carrier pins) may increase a torque/force capacity of the system.

FIG. 19C shows an exploded view of the second end cap 344 and the housing 340. As described herein, the housing 340 may include one or more openings 334 (one of which is labeled) and each carrier pin 330 may extend into and be supported by the housing 340.

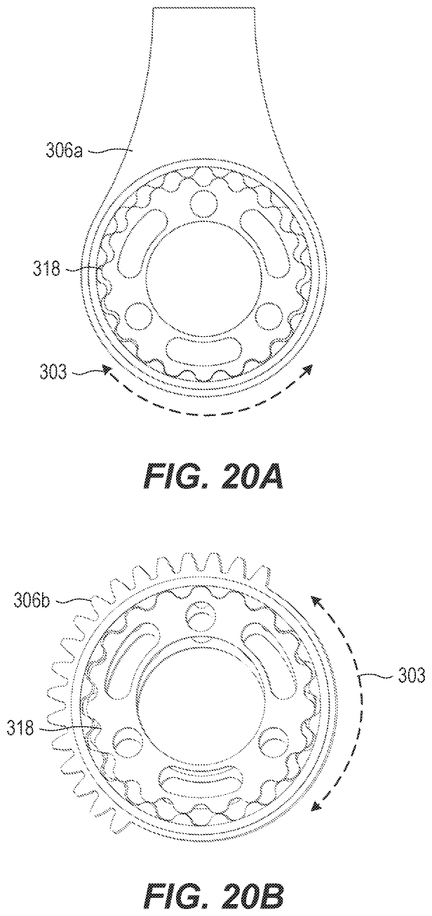

FIG. 20A shows an example of the output flange 306a and the cycloidal disks 318. As described herein, the cycloidal disks can cause output flange 306a to rotate either direction along path 303. The cycloidal disks 318 may eccentrically rotate and the output flange 306a may concentrically rotate. The output flange 306a can include features such as a lever arm that can interface with the first phalange (e.g., first phalange 104 or first phalange 704) to cause movement of the prosthetic finger as descried herein.

FIG. 20B shows an example of the output gear 306b and the cycloidal disks 318. In some cases, the output gear 306b may include gear teeth around the entire circumference of the output gear 306b or one or more portions of the circumference of the output gear 306b. The gear teeth can be configured as any suitable gear design such as spur gear, helical, worm and so on. The gear teeth can interface with gear teeth and/or other features on the first phalange (e.g., first phalange 104 or first phalange 704) to cause movement of the prosthetic finger as descried herein.

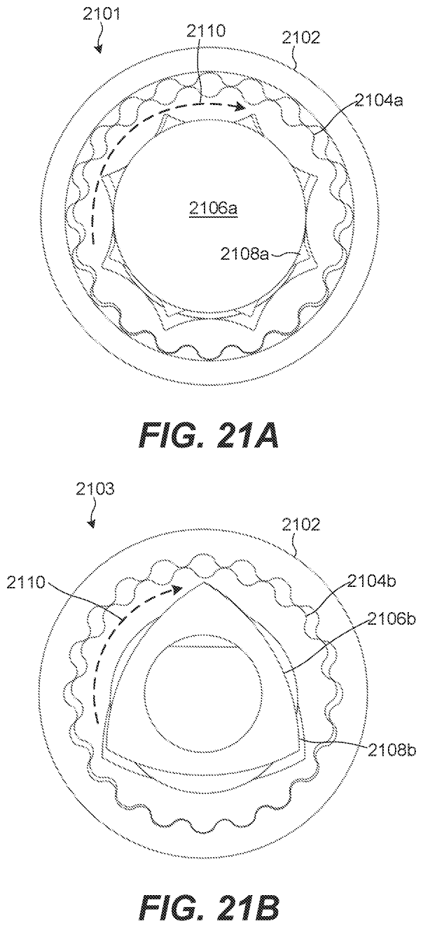

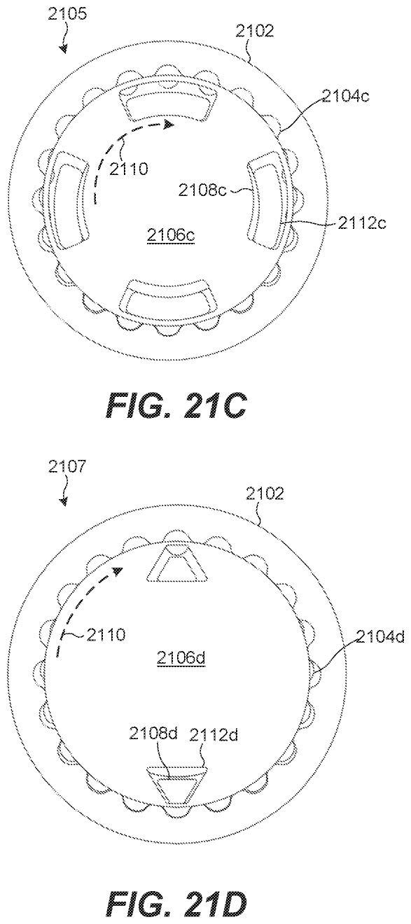

FIGS. 21A-21D show additional examples of cycloidal gear configurations that can be used in a powered prosthetic finger. In some cases, the different designs may be used to increase a shearing area of an interface between the cycloid disk and the output shaft, which can increase the load capacity of the gear system (e.g., gear system 202). The example cycloidal gear configurations shown in FIGS. 21A-21D can be implement at the first stage (S1) cycloidal gear components (e.g., the first cycloidal disk 212 and the first output shaft) and/or at the second stage (S2) cycloidal gear components (e.g., the second and third cycloidal disks 218 and the output shaft 206). In some cases, the cycloidal gear configurations shown in FIGS. 21A-21D may include a single cycloidal disk at the first stage (S1) cycloidal gear and/or the second stage (S2) cycloidal gear. The example cycloidal gear configurations described herein are provided to illustrate the concepts and are not limiting. Accordingly, other cycloidal gear configurations are possible and can be integrated in the gear systems described herein.

FIG. 21A shows an example of a first cycloidal gear configuration 2101 that has a star profile. The first cycloidal gear configuration 2101 can include a housing 2102, which can correspond to the housing 240 and defines a set of internal lobes. The first cycloidal gear configuration 2101 can include a first cycloidal disk 2104a that defines a set of external lobes that interface with the lobes on the housing 2102 as described herein. The first cycloidal disk 2104a can also define an internal (star) profile. The first cycloidal gear configuration 2101 can also include a first cam 2106a that defines an external (star) profile that interfaces with the internal star profile on the first cycloidal disk 2104a. The external star profile can include first lobes 2108a (one of which is labeled for clarity) that engage with the star profile defined by the first cycloidal disk 2104a. As the first cycloidal disk 2104a is driven (e.g., by the eccentric shaft as described herein) rotational motion of the first cycloidal disk 2104a in a first direction 2110 can cause the first cam 2106a to rotate in the first direction 2110 (e.g., by shearing effect of the internal and external start profiles). In other cases, the first cam 2106a be coupled to the input gear (e.g., the first gear via the input shaft) and the first cycloidal disk 2104a can be coupled to the output shaft.

FIG. 21B shows an example of a second cycloidal gear configuration 2103 that has a triangular profile. The second cycloidal gear configuration 2103 can include the housing 2102 that defines a set of internal lobes. The second cycloidal gear configuration 2103 can include a second cycloidal disk 2104b that defines a set of external lobes that interface with the lobes on the housing 2102 as described herein. The second cycloidal disk 2104b can also define an internal (triangular) profile. The second cycloidal gear configuration 2103 can also include a second cam 2106b that defines an external (triangular) profile that interfaces with the internal triangular profile on the second cycloidal disk 2104b. The external triangular profile can include second lobes 2108b (one of which is labeled for clarity) that engage with the triangular profile defined by the second cycloidal disk 2104b. As the second cycloidal disk 2104b is driven (e.g., by the eccentric shaft as described herein) rotational motion of the second cycloidal disk 2104b in the first direction 2110 can cause the second cam 2106b to rotate in the first direction 2110 (e.g., by shearing effect of the internal and external start profiles). In other cases, the second cam 2106b can be coupled to the input gear (e.g., the first gear via the input shaft) and the second cycloidal disk 2104b can be coupled to the output shaft.

FIG. 21C shows an example of a third cycloidal gear configuration 2105 that has a rectangular pin profile. The third cycloidal gear configuration 2105 can include the housing 2102 that defines a set of internal lobes. The third cycloidal gear configuration 2105 can include a third cycloidal disk 2104c that defines a set of external lobes that interface with the lobes on the housing 2102 as described herein. The third cycloidal disk 2104c can also define a set of pins 2112c (e.g., rectangular pins). The third cycloidal gear configuration 2105 can also include a third cam 2106c that defines openings 2108c (e.g., rectangular opening), the pins 2112c can be positioned within the openings 2108c and engage with the openings 2108c. As the third cycloidal disk 2104c is driven (e.g., by the eccentric shaft as described herein) rotational motion of the third cycloidal disk 2104c in the first direction 2110 can cause the third cam 2106c to rotate in the first direction 2110 (e.g., by shearing effect of the pins 2112c moving along the internal surface of openings 2108c). In other cases, the third cam 2106c can be coupled to the input gear (e.g., the first gear via the input shaft) and the third cycloidal disk 2104c can be coupled to the output shaft.

FIG. 21D shows an example of a fourth cycloidal gear configuration 2107 that has a trapezoidal pin profile. The fourth cycloidal gear configuration 2107 can include the housing 2102 that defines a set of internal lobes. The fourth cycloidal gear configuration 2107 can include a fourth cycloidal disk 2104d that defines a set of external lobes that interface with the lobes on the housing 2102 as described herein. The fourth cycloidal disk 2104d can also define a set of pins 2112d (e.g., trapezoidal pins). The fourth cycloidal gear configuration 2107 can also include a fourth cam 2106d that defines openings 2108d (e.g., trapezoidal openings), the pins 2112d can be positioned within the openings 2108d and engage with the openings 2108d. As the fourth cycloidal disk 2104d is driven (e.g., by the eccentric shaft as described herein) rotational motion of the fourth cycloidal disk 2104d in the first direction 2110 can cause the fourth cam 2106d to rotate in the first direction 2110 (e.g., by shearing effect of the pins 2112d moving along the internal surface of openings 2108d). In other cases, the fourth cam 2106d can be coupled to the input gear (e.g., the first gear via the input shaft) and the third cycloidal disk 2104c can be coupled to the output shaft.

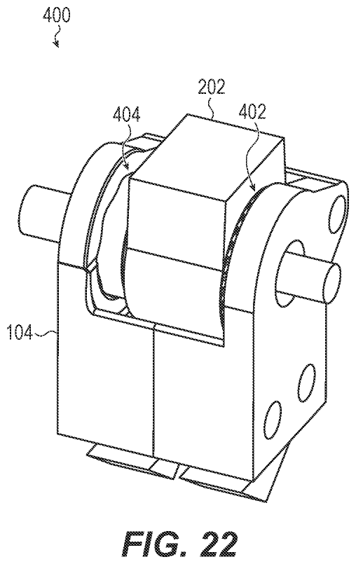

FIG. 22 shows an example clutch system 400 that can be used with the prosthetic fingers described herein, such as prosthetic fingers 100 or 700. The clutch system 400 can engage in response to a threshold force (e.g., torque) being applied to the prosthetic finger 100 and can lock the gear system 202 housing to the first phalange 104, which can cause the prosthetic finger 100 to lock in place. The locking of the gear system 202 housing to the first phalange 104 can cause forces applied to the prosthetic finger 100 to be substantially transferred through the gear system 202 housing 401 and the phalanges, which can protect the gear system 202 from damage. Accordingly, the clutch system 400 may increase forces that can be applied to a prosthetic finger (e.g., prosthetic fingers 100, 700). For example, the clutch system 400 may enable the prosthetic finger 100 to sustain forces during a static gripping operation that would otherwise damage internal components of the gear system 202.

The clutch system 400 can include multiple components that function together to engage and disengage the gear system 202 housing with the first phalange 104. The clutch system 400 can include locking features 402 on the gear system 202 housing 401 and the first phalange 104 that when engaged fix the gear system 202 housing (and second phalange) with respect to the first phalange 104. The clutch system 400 can also include an engagement system 404 that causes the locking features 402 on the gear system 202 housing and the first phalange 104 to engage and disengage.

The engagement system 404 can be configured to cause engagement of the locking features 402 in response to a force on the clutch meeting a threshold. For example, if the torque on the output shaft of the gearbox hits a torque threshold, the engagement system 404 can cause the locking features 402 on the gear system 202 housing and the first phalange 104 to engage, which can fix the gear system 202 housing (and second phalange) with respect to the first phalange 104. In the engaged/fix state, torque applied to the gear system 202 may be primarily transferred through the gear system 202 housing, which can protect the internal gear components by reducing/capping the torque (or other forces) that are applied to these components when the locking system is engaged.

In some cases, the engagement system 404 can cause disengagement of the locking system in response to force on the gear system (e.g., gear system 202) dropping below the threshold. In other cases, the drive system may be driven to release the clutch system 400 and cause disengagement of the locking features 402.

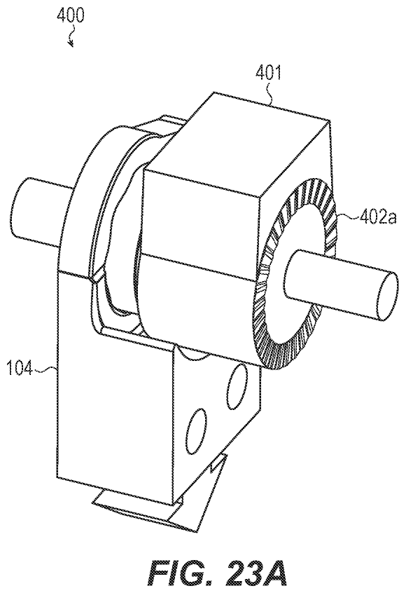

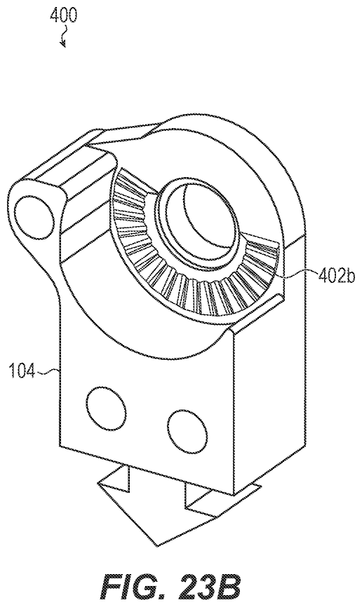

FIG. 23A shows a perspective view of the clutch system 400 with a first portion of the first phalange 104 removed. In some cases, the first phalange 104 can include multiple body segments that are coupled together as described herein. that are coupled together. The locking features 402 can include a first locking features 402a that is defined by the gear system 202 housing 401. The first locking features 402a can be a set of ratchet features, gear features, or any other suitable configuration. In other cases, the first locking features 402a can be coupled to the housing 401, for example the first locking features 402a can be formed on another component and coupled to the housing 401 using fasteners.

FIG. 23B shows a perspective view of the clutch system 400 with a second portion of the first phalange 104 removed. The locking features 402 can include second locking features 402b that are defined by the first phalange 104. The second locking features 402b can be a set of ratchet features, gear features or any other suitable configuration that engages with the first locking features 402a and prevents rotational movement of the housing 401 with respect to the first phalange 104. In other cases, the second locking features 402b can be coupled to the housing 401, for example, the second locking features 402b can be formed on another component and coupled to the housing using fasteners.

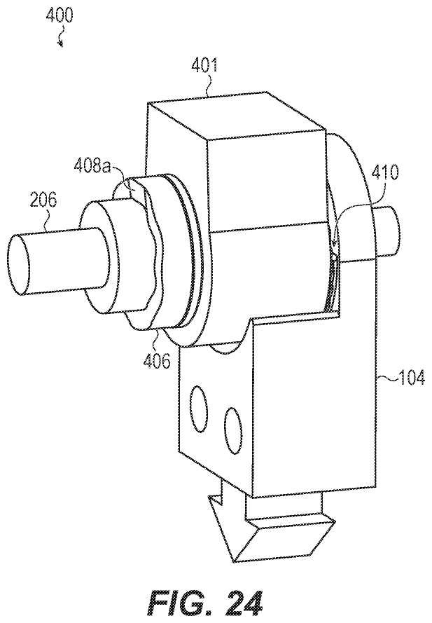

FIG. 24 shows a perspective view of the clutch system 400 with the second portion of the first phalange 104 removed. The clutch system 400 can include a first engagement member 406 that is coupled to the output shaft 206 of the gear system 202 and defines first wave features 408a. The first engagement member 406 can define the first wave features 408a in an annular configuration that rotate concentrically with the output shaft 206. The first wave features 408a can be a set of alternating peaks and valleys defined by the surface of the first engagement member 406.

FIG. 25 shows a perspective view of the clutch system 400 with the gear system 202 and the first portion of the first phalange 104 removed. The engagement system 404 can include second wave features 408b that are defined by the first phalange 104. The second wave features 408b may be in an annular or semi-annular configuration that is concentric about the axis of rotation of the output shaft 206. The first wave features 408a and the second wave features 408b can be configured to engage such that the peaks of the first wave features 408a are positioned within the valleys of the second wave features 408b and vice versa.

As illustrated in FIG. 24, the clutch system 400 can include a biasing member 410 that biases the first set of wave features 408a to engage peak to valley with the second set of wave features 408b. The biasing force of the biasing member 410 can be configured to set the force that causes the clutch to engage, as described herein. The biasing member 410 can be a spring washer such as Belleville washer, a helical spring, a compressible material, and/or any other suitable component that provides a biasing force against the gear system 202 housing 401.

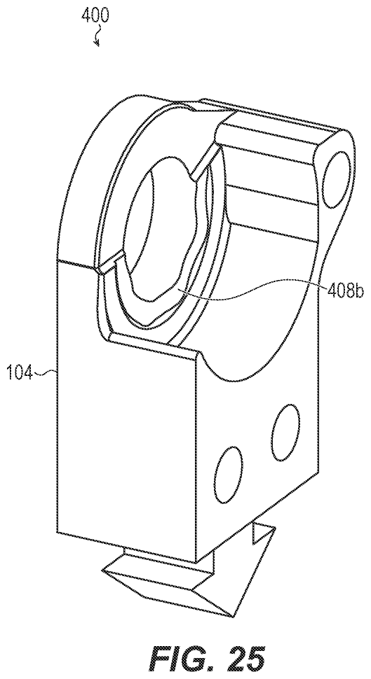

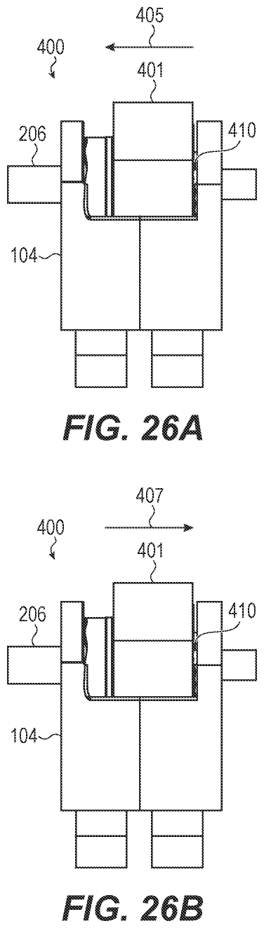

FIGS. 26A and 26B show an example operation of the clutch system 400. FIG. 26A shows the clutch system 400 in a disengage state. In the disengaged state, the gear system 202 housing 401 is biased in a first direction 405, the first locking features 402a are disengaged from the second set of locking features 402b and the gear system housing 401 (and second phalange) can move with respect to the first phalange 104. FIG. 26B shows the clutch system 400 in the engaged state. In the engaged state, the gear system housing 401 is biased in the second direction 407, the first locking features 402a are engaged with the second locking features 402b and the gear system housing 401 (and second phalange) are fixed with respect to the first phalange 104.

When the force on the output shaft 206 (e.g., torque on the output shaft) is below a threshold, the biasing member 410 causes the first wave features 408a (shown in FIG. 24) to engage peak-to-valley with the second wave features 408b (shown in FIG. 25). When the first set of wave features 408a is engaged with the second set of wave features 408b, the gear system housing 401 is shifted in the first direction 405 thereby causing the first locking features 402a to disengage from the second set of locking features 402b.

When the force on the output shaft 206 meets the threshold, the first set of wave features 408a can move from the peak-to-valley configuration with the second set of wave features 408b and toward a peak-to-peak configuration. The movement toward the peak-to-peak configuration compresses the biasing member 410 and shifts the gear system housing 401 in the second direction, which causes the first locking features 402a to engage with the second set of locking features 402b.

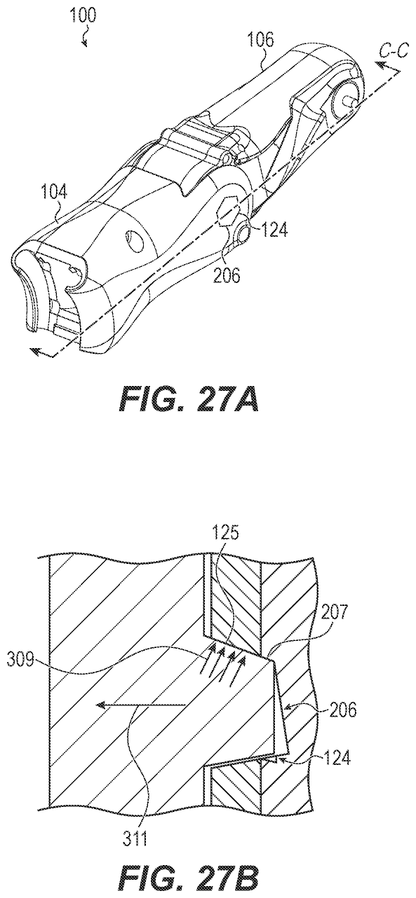

FIGS. 27A and 20B show an example of an output shaft configuration that can be used to control engagement of the clutch system, such as the clutch system 400. FIG. 27B shows a section view, taken along line C-C, of the coupling of the output shaft 206 to the opening 124. As described herein, the output shaft 206 of the gear system can be keyed and engaged with a keyed opening 124 on the first phalange 104. In some cases, the keyed opening 124 may a straight opening and the walls defining the keyed opening 124 may be substantially parallel to the walls of the output shaft 206.

In some cases, the keyed opening 124 and the output shaft 206 can be configured to control engagement of the clutch system 400. For example, as an alternative to or in addition to the wave features 408 of the engagement system 304, the output shaft 206 and the keyed opening 124 can be configured to cause the gear system housing to move between engaged and disengaged positions. As shown in FIG. 20B, the keyed opening 124 can define one or more first angled surfaces 125 and the output shaft 206 can define one or more second angled surfaces 207 that contact the first angled surfaces 125. Torque between the output shaft 206 and the first phalange 104 can generate a force 309 that partially acts in a first direction 311. The force 309 can cause movement of the gear system housing in the first direction 311 and causes the locking system (e.g., locking features 402) to engage as described herein. The slope, size and/or other parameters of the first and second angled surfaces 124, 207 can be configured to set a threshold force that causes movement of the gear system housing and engagement of the locking features, as described herein.

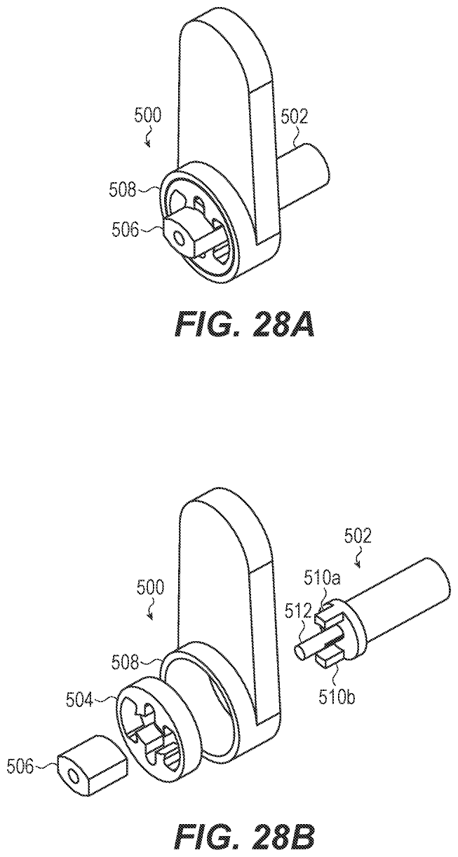

FIG. 28A shows an example clutch system 500 for a prosthetic finger (e.g., prosthetic finger 100). The clutch system 500 can include an output shaft 502, which can be an example of the output shafts described herein (e.g., output shaft 206); a flexure element 504; a clutch shaft 506; and a housing 508, which may be the housing of the second phalange 106 as described herein. The output shaft 502 can be part of the gear system (e.g., gear system 202) and rotate in response to a motor output as described herein. The output shaft 502 can be coupled to the flexure element 504 and the clutch shaft 506. When the clutch system 500 is disengaged, the output shaft 502, the flexure element 504, and the clutch shaft 506 may rotate with respect to the housing 508. When a torque differential between the clutch shaft 506 and the output shaft meets a threshold, relative movement of the output shaft 502 and the clutch shaft 506 can deform the flexure element 504 causing it to contact the housing 508. The contact of the flexure element 504 with the housing 508 can cause torque on the clutch shaft to be primarily transferred to the housing 508, which may help protect internal components of the gear system as described herein.

FIG. 28B shows an exploded view of the clutch system 500 shown in FIG. 21A. The output shaft 502 can include a central shaft 512 and cams 510. The central shaft 512 can couple to the clutch shaft 506 and the cams 510 can be positioned within cam openings 514 defined by the flexure element 504. The clutch shaft 506 can be positioned partially within the flexure element 504 and extend partially out of the flexure element 504. The portion of the clutch shaft 506 that extends out of the flexure element 504 can couple to the first phalange, for example, and extend through a keyed opening (e.g., opening 124) as described herein. Accordingly, rotation of the output shaft 502 can cause rotation of the clutch shaft 506 thereby causing extension and flexion of a prosthetic finger as described herein.

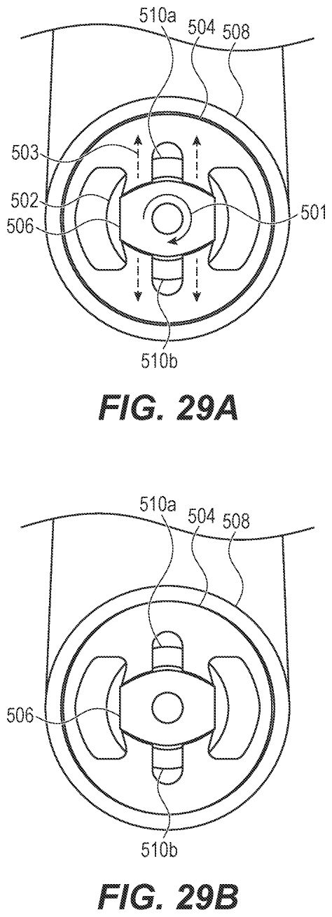

FIG. 29A shows the clutch system 500 in a disengaged state. When integrated into a prosthetic finger, the clutch shaft 506 can be anchored to a first phalange (e.g., the first phalange 104) and the output shaft 502 can be anchored to a second phalange (e.g., the second phalange 106). The clutch shaft 506 can be configured to rotate with respect to the output shaft 502. The flexure element 504 can be configured to couple the rotation of the output shaft 502 and the clutch shaft 506.

When a force is applied to the prosthetic finger, the output shaft 502 will experience a first torque (grounded to the second phalange) and the clutch shaft 506 will experience a second torque (grounded to the first phalange). This differential torque can create deformation 503 in the flexure element 504 due to the differential motion of the cams 510 on the output shaft 502 and the clutch shaft 506.

When a difference between the first torque and the second torque is below a threshold, the flexure element 504 can rotate with respect to the housing 508—i.e., deformation in the flexure element is not enough to cause it to contact the housing 508 and/or the frictional forces are low and the flexure element can rotate with respect to the housing.

As shown in FIG. 29A, when a difference between the first torque and the second torque meets a threshold, the flexure element 504 will deform enough to contact the housing 508 and reduce and/or prevent rotation of the output shaft 502 with respect to the housing. The clutch system 500 can be in an engaged state when the flexure element is deformed enough to contact the housing with sufficient force to prevent rotation of the output shaft 502. In the engaged state, forces exerted on the prosthetic finger may be primarily transferred to the housing 508 via the clutch shaft 506 and the flexure element 504, which may help protect internal components of the gear system as described herein. The properties of the clutch system 500, such as the stiffness and/or design of the flexure element 504, can be controlled to set a threshold force that causes engagement and/or disengagement of the clutch system 500. In other examples, an outer profile of the flexure element 504 can include features such as a set of lobes or teeth and the inner profile of the housing 508 can include mating lobes or teeth. In these examples, as the flexure element 504 expands, the lobes/teeth on the flexure element 504 can engage with the lobes/teeth on the housing 508. The lobes/teeth may provide discrete locking positions and/or increase a force that can be withstood by the clutch system 500.

In some cases, a prosthetic finger, such as described herein, can include a clutch mechanism that may help protect a drive system (e.g., the gear system and/or motor) from damage due to forces that are applied to the prosthetic finger. In some cases, the clutch mechanism may allow a prosthetic finger to hold a greater amount of static weight by redirecting/transferring force applied to the prosthetic system away from the gear system.

Generally and broadly, the clutch examples described with respect to FIGS. 29-37 include clutch designs that use various cantilevered mechanism to direct/transfer force applied to a prosthetic finger away from the drive system. For example the cantilevered clutch mechanisms described herein may engage in response to a threshold amount of force being applied to a prosthetic system and direct the force into the housing components. In some cases, the clutch mechanism can be applied when the drive system is in a static state (e.g., not being driven).

Embodiments of a prosthetic finger that include a cantilever clutch mechanism may include one or more cantilevered structures that couple the drive system to a driven phalange. For example, a first portion of the cantilevered structures may couple to the drive system 200 or 300 and a second portion of the cantilevered structures may couple to the first phalange 104, 704, or 804. The cantilevered structure may include first grounding features (e.g., gear teeth) that do not engage with corresponding second grounding features (e.g., gear teeth) on the driven link (e.g., the first phalange), when a force applied to the prosthetic finger is below a threshold. In response to a force above a threshold being applied to the prosthetic finger (e.g., causing a threshold amount of torque between the first phalange and the second phalange/gear system), the cantilevered structure may bend or otherwise deform and engage the first grounding features with the second grounding features. The engagement of the grounding features may temporality lock the prosthetic finger in a fixed position (e.g., lock the second phalange to the first phalange) while the force is above the threshold. This may cause applied force to be concentrated on the frame components of the prosthetic finger and help protect the gear system from damage. Once the applied force decreases below the threshold, the clutch system may disengage (e.g., the cantilevered member returns to its neutral state) and the prosthetic finger may be driven by the gear system.

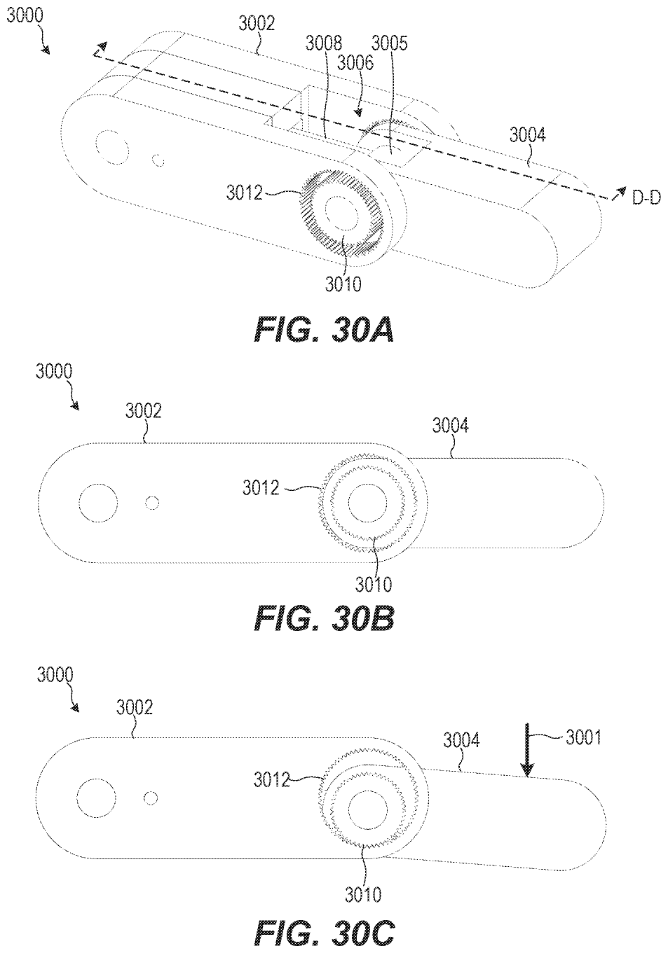

FIGS. 30A-30C show an example of a prosthetic finger 3000 that includes a cantilevered clutch mechanism. The prosthetic finger 3000 can be an example of the prosthetic fingers 100, 700 and 800 described herein. The example shown in FIG. 30 can include a representative first phalange 3002, a representative second phalange 3004, which may be example of the phalanges described herein. The second phalange 3004 may include a drive system 3005, which may be an example of the drive systems described herein.

The prosthetic finger 3000 can include clutch mechanism 3006 that includes a cantilevered member 3008 that couples to the first phalange 3002 and to the drive system 3005. In some cases, the cantilevered member 3008 may be rigidly coupled to the first phalange 3002 and the drive system 3005 (and/or the second phalange 3004). In some cases, the cantilevered member 3008 may be formed as part of the first phalange 3002, the second phalange 3004 or the drive system 3005.

The clutch mechanism 3006 can also include a first set of grounding features 3010 (e.g., gear teeth) that are coupled to the second phalange 3004 and/or drive system 3005, and a second set of grounding features 3012 (e.g., gear teeth) that are coupled to the first phalange 3002.

FIG. 30B shows an example of the prosthetic finger when an applied force is below a force threshold (e.g., no applied force). When the applied force is below the force threshold, the first set of grounding features 3010 do not engage with the second set of grounding features 3012. For example, the first set of mounting features may be formed on a portion of the second phalange 3004/drive system that is offset from an opening defined by the first phalange 3002. Accordingly, the first set of grounding features 3010 may rotate with respect to the second set of grounding features 3010, for example, as the second phalange 3004 rotates with respect to the first phalange 3002 (e.g., in extension or flexion).

FIG. 30C shows an example of the prosthetic finger when an applied force 3001 is above a force threshold. The applied force may cause the cantilevered member 3008 to bend causing the first set of grounding features 3010 to engage with the second set of grounding features 3012, which can lock the rotation of second phalange 3004 with respect to the first phalange 3002. The engagement of the first set of grounding features 3010 with the second set of grounding features 3012, may cause applied forces (e.g., the force 3001) to be primarily transferred to the housing components of the prosthetic finger and away from the drive system.

The force threshold may be defined by the design of the clutch mechanism. For example, the cantilevered member 3008 may be designed to bend and engage the first grounding features 3010 with the second grounding features 3012 at a predetermined force level (e.g., a defined amount of torque between the first phalange 3002 and the second phalange 3004/drive system). For example, the shape, size, material (e.g., rigidity) and/or any other suitable property of the clutch mechanism (e.g., the cantilevered member 3008) may be configured so that the first grounding features 3010 engage with the second set of grounding features 3012 when a force at or around the force threshold is applied to the prosthetic finger. For example, when an applied force causes a threshold torque differential between the first phalange and the second phalange.

In some cases, the clutch mechanism can be bidirectional. For example, the clutch mechanism may engage in response to a torque being applied in the flexion direction or engage in response to a force being applied in the extension direction.

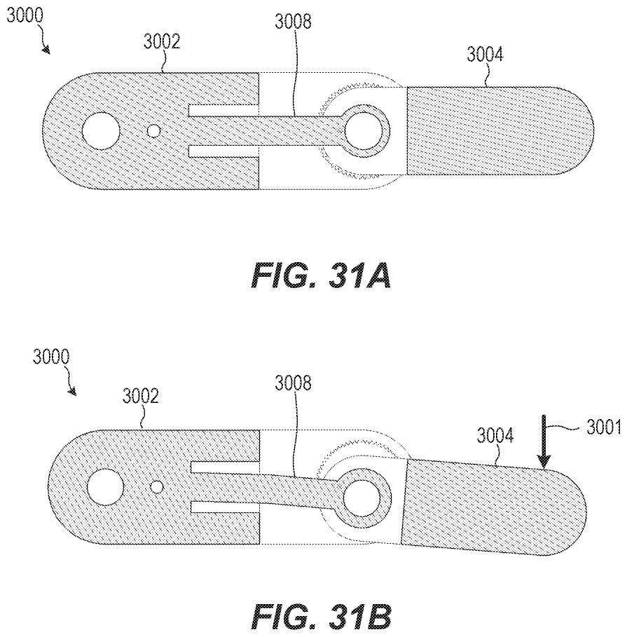

FIGS. 31A and 31B show cross-sectional views, taken along section D-D, of an example of the cantilevered clutch mechanism shown in FIGS. 30A-30C. In the example shown in FIGS. 30A and 30B, the cantilevered member may bend or otherwise deform in response to applied forces. The cantilevered member 3008 maybe part of the first phalange 3002 (e.g., formed as part of one or more housing components), formed as part of the second phalange, and/or as part of the drive system. The coupling between the cantilevered member 3008, the first phalange 3002, the second phalange 3004 and/or the drive member can cause the cantilevered member 3008 to bend or otherwise deform in response to an applied force (e.g., the force 3001).

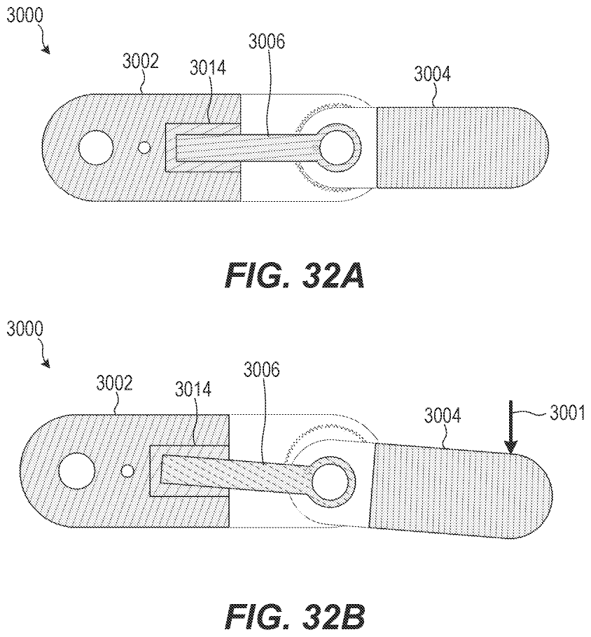

FIGS. 32A and 32B show cross-sectional views, taken along section D-D, of an example of the cantilevered clutch mechanism shown in FIGS. 30A-30C. In the example shown in FIGS. 31A and 31B, the clutch system can include a compliant member 3014 that deforms in addition to or as an alternative to the cantilevered member 3008. For example, the compliant member 3014 may include an elastically deformable materials such as a polymer that compresses in response to the force 3001 and causes the first set of grounding features 3010 to engage with the second set of grounding features 3012, as described herein.

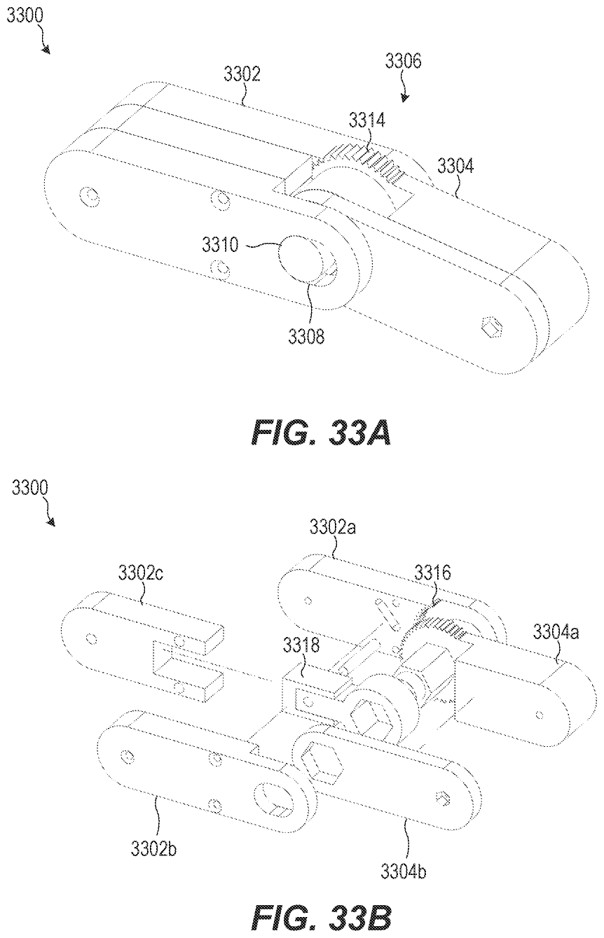

FIGS. 33A and 33B show an example of a prosthetic finger 3300 that includes a slotted clutch mechanism 3306. The prosthetic finger 3300 can be an example of the prosthetic fingers 100, 700 and 800 described herein. The example shown in FIG. 33 can include a representative first phalange 3302, a representative second phalange 3304, which may be example of the phalanges described herein. The second phalange 3304 may include a drive system, which may be an example of the drive systems described herein.

The clutch mechanism 3306 can include a slot 3308 and a pin 3310 that moves within the slot 3308. In the illustrated example, the slot is incorporated into the first phalange 3302 and the pin 3310 can be incorporated into the second phalange 3304 and/or drive system (e.g., drive system housing). The clutch mechanism may also include a first set of grounding features 3314 that are coupled to the second phalange 3304. The example shown in FIG. 33A, shows the clutch in an engaged state. For example, when an applied force is above the force threshold, the pin 3310 may move along the slot 3308 and the first set of grounding features 3314 engage with the first phalange 3302 to prevent rotation between the first phalange 3302 and the second phalange 3304, and allow applied forces to be primarily transferred to housing components, as described herein.

FIG. 33B shows an exploded view of the prosthetic finger 3300. The first phalange may include a first side component 3302a, a second side component 3302b and a middle component 3302c, which engages with cantilevered component 3318. The cantilever component may rigidly couple to the first phalange 3302 (e.g., the middle component 3302c) and rigidly couple to the second phalange 3304. For example, the second phalange may include a keyed feature that extends between a fourth side component 3304a and a fifth side component 3304b and engages with the cantilevered component. In some cases, the cantilevered component 3318 can be configured to bend so that the first set of grounding features 3314 engage with a second set of grounding features 3316 to lock the prosthetic finger 3300. Additionally or alternatively, the cantilevered component 3318 can include a compliant material such as a polymer sleeve that compresses or otherwise deforms in response to a sufficient applied force to the prosthetic finger 3300.

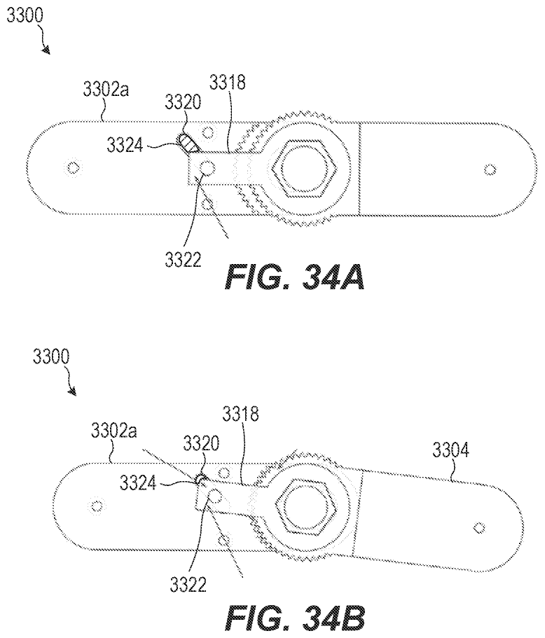

FIGS. 34A and 34B show example operation of the clutch mechanism 3306 shown in FIGS. 33A and 33B. In some cases, the clutch mechanism 3306 can include a second slot 3320 in the first phalange 3302 (e.g., the first side component 3302a) and a second pin 3322 in the cantilevered member 3318. FIG. 33A shows the prosthetic finger 3300 in a state where the clutch is not engaged (e.g., applied force is below a threshold force) and FIG. 33B shows the prosthetic finger in a state where the clutch is engaged (e.g., the applied force is above a threshold force).

The second slot 3320 and the second pin 3322 may include an elastic insert 3324 that is compressed in response to the clutch 3306 engaging and provides a force that biases the clutch to an unengaged state. In some cases, the threshold at which the pin begins to move in the slot may be determined based on the angle of the second slot 3320, which may be used to set a threshold force that causes the clutch 3306 to engage and/or disengage.

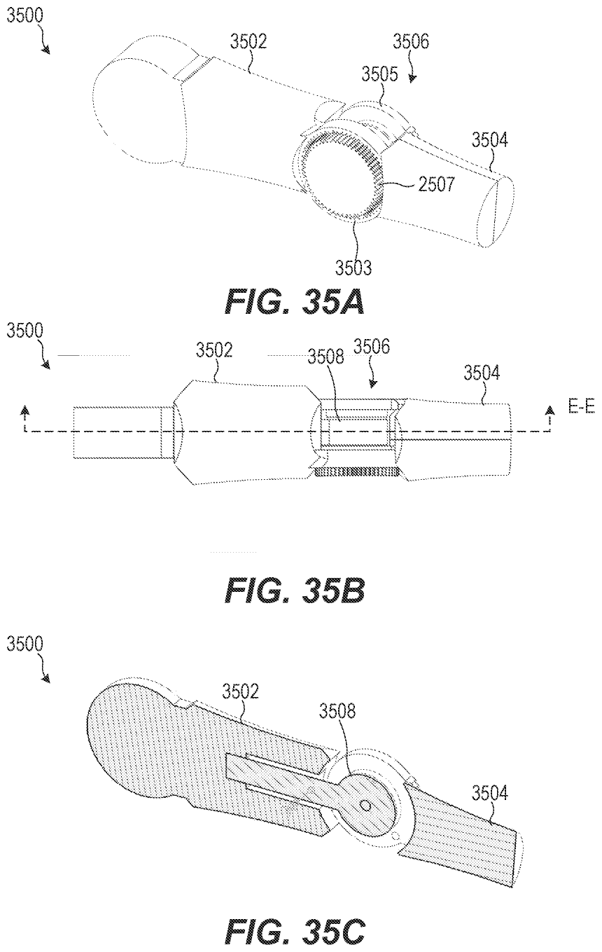

FIGS. 35A-35C show an example of a prosthetic finger 3500 that includes a cantilevered clutch mechanism 3506. FIG. 35C shows a cross-section view taken along line E-E. The prosthetic finger 3500 can be an example of the prosthetic fingers 100, 700 and 800 described herein. The example shown in FIG. 35 can include a representative first phalange 3502, a representative second phalange 3504, which may be example of the phalanges described herein. The second phalange 3504 may include a drive system, which may be an example of the drives systems described herein.

The first phalange 3502 may define a first side component that include a first set of grounding features 3503. The second phalange may define a second side component 3505 that includes a second set of grounding features. A cantilevered member 3508 may extended between the first side component and the second side component 3505. The design shown in FIGS. 35A-35C allow the cantilevered member 3508 to be positioned centrally within the prosthetic finger 3500.

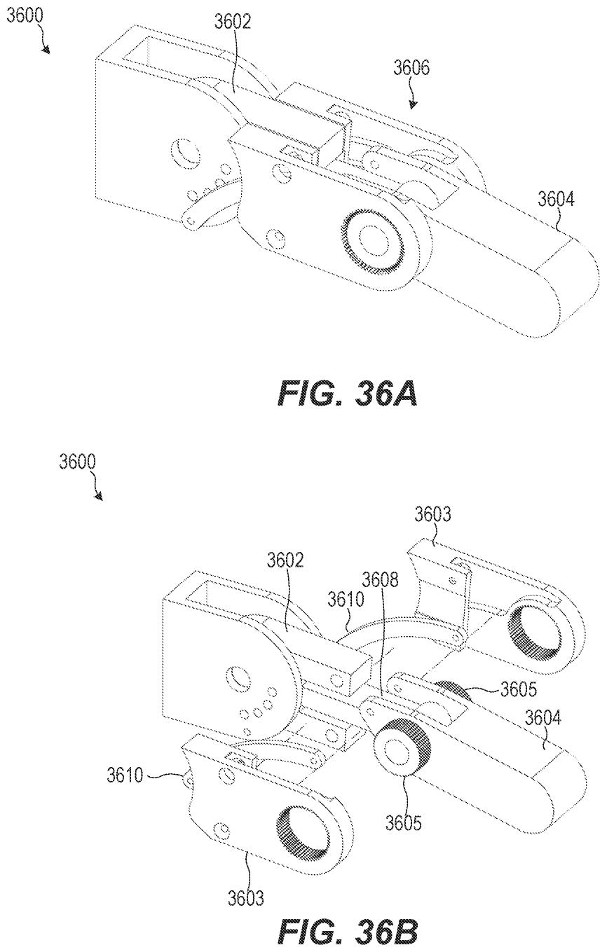

FIGS. 36A and 36B show an example of a prosthetic finger 3600 that includes a cantilevered clutch mechanism 3606. The prosthetic finger 3600 can be an example of the prosthetic fingers 100, 700 and 800 described herein. The example shown in FIG. 36 can include a representative first phalange 3602, a representative second phalange 3604, which may be example of the phalanges described herein. The second phalange 3604 may include a drive system, which may be an example of the drive systems described herein.

The clutch mechanism 3606 may include a cantilevered member 3608, as described herein. First and second side components 3603 may each rigidly couple to the first phalange 3602 and each define a first set of grounding features. The second phalange 3404 may include second grounding features 3605 which may be positioned within the first grounding features, as described herein. The clutch mechanism may include one or more links 3610 may constrain movement of the first phalange 3602 with respect to the second phalange 3604 as the cantilevered member 3608 is deformed in response to an applied force.

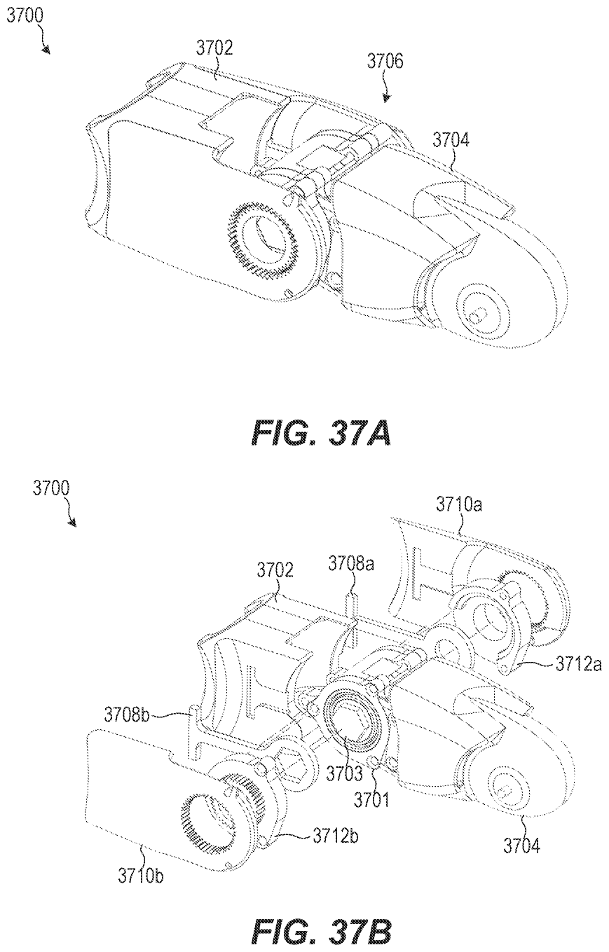

FIGS. 37A and 37B show an example of a prosthetic finger 3700 that includes a cantilevered clutch mechanism 3706. The prosthetic finger 3700 can be an example of the prosthetic fingers 100, 700 and 800 described herein. The example shown in FIG. 37 can include a representative first phalange 3702, a representative second phalange 3704, which may be example of the phalanges described herein. The second phalange 3704 may include a drive system 3701, which may be an example of a drive systems described herein.

The prosthetic finger 3700 may include a first cantilevered member 3708a which couples to a first side of the first phalange 3702 (e.g., via a keyed attachment features) and a second cantilevered member 3708b which coupled to a second side of the first phalange 3702 (e.g., via a keyed attachment features). The clutch mechanism 3706 may include a first side plate 3710a that defines a first set of grounding features and a second side plate 3710b that defines a second set of grounding features. The first and second side plates 3710 may rigidly couple to the first phalange 3702 (e.g., via a keyed attachment features). The clutch mechanism 3706 may also include a third grounding structure 3712a and a fourth grounding structure 3712b, which each rigidly couple to the second phalange 3704.

The drive system 3701 can include an output drive 3703 that couples to the second cantilevered member 3708b. The output drive 3703 may be an example of the gear system outputs described herein and rotate in response to the motor being driven.

As described herein, when an applied force on the prosthetic finger is below a force threshold, the second grounding features may be suspending within the first grounding features but remain unengaged from the first grounding features allowing the second phalange 3704 to rotate with respect to the first phalange 2702 (e.g., in response to rotation of the output drive 3703). When an applied force on the prosthetic finger is above a force threshold (e.g., causing a differential torque between the first phalange 3702 and the second phalange 3704 to exceed a threshold), the cantilever members 3708 can bend causing the second grounding features to engage with the first grounding features locking the finger and distributing the applied force (e.g., torque) to the housing components, as described herein.

The following clauses further describe various embodiments that may include various features as described above and/or illustrated in the figures:
Clause 1. A prosthetic finger comprising:
   a track that couples to a hand;
   a first phalange coupled to the track at a first joint;
   a second phalange coupled to the first phalange at a second joint;
   a third phalange coupled to the second phalange at a third joint;
   a motor positioned at least partially within the second phalange;
   a first linkage positioned at least partially within the first phalange and configured to:
      cause the first phalange to move with respect to the track in response to a motor output; and
      cause the second phalange to move with respect to the first phalange in response to the motor output; and
   a second linkage coupling the third phalange to the first phalange and configured to cause the third phalange to move with respect to the second phalange in response to the motor output.
Clause 2. The prosthetic finger of clause 1, wherein:
   the motor is configured to produce a first motor output having a first direction and a second motor output having a second direction;
   in response to the first motor output, the first phalange, the second phalange, and the third phalange move in a flexion direction; and
   in response to the second motor output, the first phalange, the second phalange, and the third phalange move in an extension direction.
Clause 3. The prosthetic finger of clause 2, wherein:
   in response to the first motor output, the first phalange moves along the track in the first direction; and
   in response to the second motor output, the first phalange moves along the track in the second direction.
Clause 4. The prosthetic finger of clause 1, wherein:
   the first linkage comprises:
      a first link bar having a first end coupled to the track;
      a second link bar having a first end coupled to the first phalange; and
      a third link bar having a first end coupled to the second phalange; and
   second ends of the first link bar, the second link bar, and the third link bar are coupled to each other.
Clause 5. The prosthetic finger of clause 4, wherein:
   the first phalange defines an interior cavity; and
   the second ends of the first link bar, the second link bar, and the third link bar remain positioned within the interior cavity through a full range of motion of the prosthetic finger.
Clause 6. The prosthetic finger of clause 4, wherein the first link bar, the second link bar, and the third link bar are each curved along a respective length.
Clause 7. The prosthetic finger of clause 1, further comprising a gear system coupled to the motor, wherein the gear system couples the second phalange to the first phalange and the second joint.
Clause 8. The prosthetic finger of clause 7, wherein:
   the gear system comprises an output shaft that couples to the first phalange; and
   the motor output causes the output shaft to rotate the first phalange with respect to the second phalange.
Clause 9. The prosthetic finger of clause 7, wherein the second linkage comprises:
   a first link bar that extends along a first side of the second phalange and comprises a first end coupled to the first phalange and a second end coupled to the third phalange; and
   a second link bar that extends along a second side of the second phalange and comprises a first end coupled to the first phalange and a second end coupled to the third phalange.
Clause 10. The prosthetic finger of clause 9, wherein:
   the second phalange defines a first channel along the first side of the second phalange;
   the first link bar is positioned at least partially within the first channel;
   the second phalange defines a second channel along the second side of the second phalange; and
   the second link bar is positioned at least partially with the second channel.
Clause 11. A prosthetic device comprising:
   a track that couples to a hand;
   a first phalange coupled to the track at a first joint;
   a second phalange coupled to the first phalange at a second joint;
   a motor positioned at least partially within the second phalange; and
   a first bar positioned at least partially within the first phalange and configured to:
      cause the first phalange to move with respect to the track in response to a motor output; and
      cause the second phalange to move with respect to the first phalange in response to the motor output.
Clause 12. The prosthetic device of clause 11, wherein:
   the track defines one or more rails;
   the first phalange comprises one or more slides that couple to the one or more rails; and
   the first phalange is configured to move along the track.
Clause 13. The prosthetic device of clause 12, wherein:
   the one or more rails define a curved profile; and
   the first phalange is configured to move along the curved profile of the one or more rails.
Clause 14. The prosthetic device of clause 11, wherein:
   the second phalange comprises:
      a body portion; and
      a gear system coupled to the body portion and operably coupled to the motor; and
   the gear system is rotatably coupled to the first phalange.
Clause 15. The prosthetic device of clause 14, wherein:
   the gear system comprises an output shaft coupled to the first phalange; and
   the output shaft causes the second phalange to move with respect to the first phalange in response to the motor output.
Clause 16. A prosthetic finger comprising:
   a track that couples to a hand;
   a first phalange coupled to the track at a first joint;
   a second phalange coupled to the first phalange at a second joint; and
   a first linkage positioned at least partially within the first phalange and comprising:
      a first link bar defining a first end coupled to the track and a second end;
      a second link bar defining a first end coupled to the first phalange and a second end; and
      a third link bar defining a first end coupled to the second phalange and second end, wherein the second ends of the first link bar, the second link bar and the third link bar are coupled together.
Clause 17. The prosthetic finger of clause 16, wherein the second ends of the first link bar, the second link bar and the third link bar are positioned within an interior of the first phalange.
Clause 18. The prosthetic finger on clause 16, further comprising:
   a third phalange coupled to the second phalange at a third joint; and
   a second linkage coupling the third phalange to the first phalange.
Clause 19. The prosthetic finger of clause 18, wherein the second linkage comprises:
   a fourth link bar that extends along a first side of the second phalange; and
   a fifth link bar that extends along a second side of the second phalange.
Clause 20. The prosthetic finger of clause 19, wherein:
   the second phalange defines a first channel;
   the fourth link bar is positioned at least partially within the first channel;
   the second phalange defines a second channel; and
   the fifth link bar is positioned at least partially within the second channel.
Clause 21. A prosthetic finger comprising:
   a first phalange that couples to a hand;
   a second phalange that couples to the first phalange at a joint;
   a motor positioned at least partially within the second phalange; and
   a cycloidal gear system comprising:
      an input gear operably coupled to the motor; and
      an output shaft coupled to the joint; wherein, in response to an output from the motor, the cycloidal gear system causes the second phalange to move with respect to the first phalange.
Clause 22. The prosthetic finger of clause 21, wherein:
   the second phalange extends between a proximal end and a distal end; and
   the cycloidal gear system is coupled to the proximal end of the second phalange.
Clause 23. The prosthetic finger of clause 22, wherein
   the motor extends between a base end and an output end;
   the base end is positioned proximate to the distal end of the second phalange; and
   the output end is positioned proximate to the proximal end of the second phalange.
Clause 24. The prosthetic finger of the clause 21, wherein:
   the motor comprises a first gear;
   the output comprises rotation of the first gear about a first axis;
   the cycloidal gear system comprises a second gear; and
   the rotation of the first gear causes rotation of the second gear about a second axis.
Clause 25. The prosthetic finger of clause 24, wherein:
   the cycloidal gear system comprises an eccentric shaft; and
   the second gear is coupled to the eccentric shaft.
Clause 26. The prosthetic finger of clause 21, wherein:
   the first phalange defines a keyed opening;
   the output shaft comprises an outer profile that corresponds to the keyed opening; and
   the output shaft extends at least partially through the keyed opening.
Clause 27. The prosthetic finger of clause 21, wherein the cycloidal gear system comprises a two-stage cycloidal gear.
Clause 28. The prosthetic finger of clause 21, wherein:
   the cycloidal gear system comprises a housing defining a first side and a second side;
   a first cycloidal gear assembly is positioned on the first side of the housing; and
   a second cycloidal gear assembly is positioned on the second side of the housing.
Clause 29. The prosthetic finger of clause 28, wherein:
   the first cycloidal gear assembly comprises at least one cycloidal disk; and
   the second cycloidal gear assembly comprises at least two cycloidal disks.
Clause 30. The prosthetic finger of clause 28, wherein:
   the cycloidal gear system further comprises a keyed shaft; and
   the first cycloidal gear assembly is coupled to the second cycloidal gear assembly by the keyed shaft.
Clause 31. A prosthetic finger comprising:
   a first phalange that couples to a hand;
   a second phalange that couples to the first phalange at a joint and comprising:
      a motor positioned at least partially within the second phalange; and
      a cycloidal gear system comprising:
         an input gear operably coupled to the motor;
         a first cycloidal gear stage driven by the input gear; and
         a second cycloidal gear stage driven by the first cycloidal gear stage; and
         an output shaft driven by the second cycloidal gear stage.
Clause 32. The prosthetic finger of clause 31, wherein the output shaft is coupled to the first phalange.
Clause 33. The prosthetic finger of clause 31, wherein:
   the first cycloidal gear stage comprises a single cycloidal disk; and
   the second cycloidal gear stage comprises two cycloidal disks.
Clause 34. The prosthetic finger of clause 31, wherein:
   the second phalange further comprises a body portion;
   the cycloidal gear system is coupled to the body portion.
Clause 35. The prosthetic finger of clause 34, wherein the second phalange is coupled to the first phalange by the cycloidal gear system.
Clause 36. A prosthetic finger comprising:
   a first phalange that couples to a hand; and
   a second phalange that couples to the first phalange and comprising:
      a body portion;
      a motor positioned at least partially within the body portion comprising an output gear; and
      a cycloidal gear system comprising:
      an input gear operably coupled to the output gear of the motor; and
      an output shaft coupled to the first phalange.
Clause 37. The prosthetic finger of clause 36, wherein the cycloidal gear system causes the first phalange to move with respect to the second phalange in response to a motor output
Clause 38. The prosthetic finger of clause 37, further comprising:
   a track that couples to a hand; and
   a linkage system coupled to the track, the first phalange and the second phalange wherein, the linkage system causes the first phalange to move along the track in response to the motor output.
Clause 39. The prosthetic finger of clause 38, further comprising a third phalange rigidly coupled to the second phalange.
Clause 40. The prosthetic finger of clause 36, wherein the cycloidal gear system comprises a multi-stage cycloidal gear.
Clause 41. A prosthetic finger comprising:
   a first phalange that couples to a hand;
   a second phalange coupled to the first phalange at a joint and comprising:
      a body portion;
      a motor positioned at least partially within the body portion; and
      a gear housing coupled to the body portion and comprising a gear system operably coupled to the motor; and
   a clutch positioned at the joint and configured to:
      in response to a force on the clutch below a threshold, cause an output of the gear system to move the first phalange with respect to the second phalange; and
      in response to the force on the clutch meeting or exceeding the threshold, cause the gear system to statically couple with respect to the first phalange and prevent movement of the first phalange with respect to the second phalange.
Clause 42. The prosthetic finger of clause 41, wherein:
   the gear system comprises first ratchet features;
   the clutch is coupled to the first phalange and comprises second ratchet features; and
   in response to the force on the clutch meeting the threshold, the first ratchet features engage with the second ratchet features.
Clause 43. The prosthetic finger of clause 42, wherein:
   the clutch comprises a compressible member positioned between the first ratchet features and the second ratchet features;
   in response to the force on the clutch below the threshold, the compressible member separates the first ratchet features from the second ratchet features; and
   in response to the force on the clutch meeting the threshold, the compressible member compresses to engage the first ratchet features with the second ratchet features.
Clause 44. The prosthetic finger of clause 43, wherein the compressible member comprises a compressible washer.
Clause 45. The prosthetic finger of clause 42, wherein:
   the clutch comprises:
      a first member coupled to the gear system defining first wave features comprising a first set of peaks and valleys; and
      second wave features defined by the first phalange and comprising a second set of peaks and valleys;
   in response to the force on the clutch below the threshold, the first set of peaks engages with the first set of valleys; and
   in response to the force on the clutch meeting the threshold, the first set of peaks contacts the second set of peaks.
Clause 46. The prosthetic finger of clause 45, wherein:
   the first and second ratchet features are positioned on a first side of the gear system; and
   the first and second sets of peaks and valleys are positioned on a second side of the gear system.
Clause 47. The prosthetic finger of clause 45, wherein:
   the gear system comprises an output shaft that rotates in response to a motor output; and
   the first member is rigidly coupled to the output shaft.
Clause 48. The prosthetic finger of clause 45, wherein:
   the gear system comprises an output shaft that rotates in response to a motor output; and
   the first member is defined by the output shaft.
Clause 49. The prosthetic finger of clause 41, wherein:
   motion of the phalanges in a first direction causes the clutch to couple the gear system to the first phalange; and
   motion of the phalanges in a second direction causes the clutch to uncouple the gear system from the first phalange.
Clause 50. The prosthetic finger of clause 41, wherein the motor is configured to cause motion in the second direction to uncouple the gear system from the first phalange.
Clause 51. A prosthetic finger comprising:
   a first phalange that couples to a hand;
   a second phalange comprising a gear system housing coupled to the first phalange to define a joint;
   a clutch comprising:
      an engagement system positioned between the first phalange and the gear system housing; and
      a locking system that, when engaged by the engagement system, is configured to prevent movement of the second phalange with respect to the first phalange.
Clause 52. The prosthetic finger of clause 51, wherein the engagement system cause the locking system to engage when a force on the prosthetic finger meets a threshold.
Clause 53. The prosthetic finger of clause 51, wherein:
   the second phalange comprises an output shaft extending from the gear system housing:
   the output shaft is coupled to the first phalange; and
   the engagement system comprises:
      a first portion coupled to the output shaft; and
      a second portion coupled to the first phalange.
Clause 54. The prosthetic finger of clause 53, wherein the engagement system further comprises a biasing member that engages the first portion with the second portion.
Clause 55. The prosthetic finger of clause 51, wherein:
   a first portion of the locking system is defined by the gear system housing; and a second portion of the locking system is defined by the first phalange.
Clause 56. A prosthetic finger comprising:
   a first phalange that couples to a hand;
   a second phalange rotatable coupled to the first phalange and defined by:
      a body portion; and
      a gear system comprising:
         a housing; and
         a first output shaft extending from the housing; and
   a clutch comprising:
      a flexure element coupled to the first output shaft;
      a second output shaft extending through the flexure element and coupled to the first output shaft and the first phalange.
Clause 57. The prosthetic finger of clause 56, wherein:
   the second phalange defined an opening; and
   the flexure element is positioned at least partially within the opening.
Clause 58. The prosthetic finger of clause 57, wherein:
   the flexure element is configured to deform in response to a difference in force on the first output shaft and the second output shaft; and
   in response to the difference in force on the first output shaft and the second output shaft meeting a threshold, the flexure element is configured to prevent movement of the first phalange with respect to the second phalange.
Clause 59. The prosthetic finger of clause 56, wherein:
   the flexure element defines one or more openings;
   the first output shaft defines:
      a central shaft; and
      one or more cams;
   the second output shaft is positioned at least partially over the central shaft; and
   the one or more cams are positioned at least partially within the one or more openings.
Clause 60. The prosthetic finger of clause 59, wherein the second output shaft can rotate with respect to the first output shaft.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A prosthetic finger comprising:
a first phalange that couples to a hand;
a second phalange coupled to the first phalange at a joint and comprising:
a body portion;
a motor positioned at least partially within the body portion; and
a gear housing coupled to the body portion and comprising a gear system operably coupled to the motor; and
a clutch positioned at the joint and configured to:
in response to a force on the clutch below a threshold, cause an output of the gear system to move the first phalange with respect to the second phalange; and
in response to the force on the clutch meeting or exceeding the threshold, cause the gear system to statically couple with respect to the first phalange and prevent movement of the first phalange with respect to the second phalange.

2. The prosthetic finger of claim 1, wherein:
the gear system comprises first ratchet features;
the clutch is coupled to the first phalange and comprises second ratchet features; and
in response to the force on the clutch meeting the threshold, the first ratchet features engage with the second ratchet features.

3. The prosthetic finger of claim 2, wherein:
the clutch comprises a compressible member positioned between the first ratchet features and the second ratchet features;
in response to the force on the clutch below the threshold, the compressible member separates the first ratchet features from the second ratchet features; and
in response to the force on the clutch meeting the threshold, the compressible member compresses to engage the first ratchet features with the second ratchet features.

4. The prosthetic finger of claim 3, wherein the compressible member comprises a compressible washer.

5. The prosthetic finger of claim 2, wherein:
the clutch comprises:
a first member coupled to the gear system defining first wave features comprising a first set of peaks and valleys; and
second wave features defined by the first phalange and comprising a second set of peaks and valleys;
in response to the force on the clutch below the threshold, the first set of peaks engages with the first set of valleys; and
in response to the force on the clutch meeting the threshold, the first set of peaks contacts the second set of peaks.

6. The prosthetic finger of claim 5, wherein:
the first and second ratchet features are positioned on a first side of the gear system; and
the first and second sets of peaks and valleys are positioned on a second side of the gear system.

7. The prosthetic finger of claim 5, wherein:
the gear system comprises an output shaft that rotates in response to a motor output; and
the first member is rigidly coupled to the output shaft.

8. The prosthetic finger of claim 5, wherein:
the gear system comprises an output shaft that rotates in response to a motor output; and
the first member is defined by the output shaft.

9. The prosthetic finger of claim 1, wherein:
motion of the phalanges in a first direction causes the clutch to couple the gear system to the first phalange; and
motion of the phalanges in a second direction causes the clutch to uncouple the gear system from the first phalange.

10. The prosthetic finger of claim 1, wherein the motor is configured to cause motion in the second direction to uncouple the gear system from the first phalange.

11. A prosthetic finger comprising:
a first phalange that couples to a hand;
a second phalange comprising a gear system housing coupled to the first phalange to define a joint;
a clutch comprising:
an engagement system positioned between the first phalange and the gear system housing; and
a locking system that, when engaged by the engagement system, is configured to prevent movement of the second phalange with respect to the first phalange.

12. The prosthetic finger of claim 11, wherein the engagement system cause the locking system to engage when a force on the prosthetic finger meets a threshold.

13. The prosthetic finger of claim 11, wherein:
the second phalange comprises an output shaft extending from the gear system housing:
the output shaft is coupled to the first phalange; and
the engagement system comprises:
a first portion coupled to the output shaft; and
a second portion coupled to the first phalange.

14. The prosthetic finger of claim 13, wherein the engagement system further comprises a biasing member that engages the first portion with the second portion.

15. The prosthetic finger of claim 11, wherein:
a first portion of the locking system is defined by the gear system housing; and
a second portion of the locking system is defined by the first phalange.

16. A prosthetic finger comprising:
a first phalange that couples to a hand;
a second phalange rotatable coupled to the first phalange and defined by:
a body portion; and
a gear system comprising:
a housing; and
a first output shaft extending from the housing; and
a clutch comprising:
a flexure element coupled to the first output shaft;
a second output shaft extending through the flexure element and coupled to the first output shaft and the first phalange.

17. The prosthetic finger of claim 16, wherein:
the second phalange defined an opening; and
the flexure element is positioned at least partially within the opening.

18. The prosthetic finger of claim 17, wherein:
the flexure element is configured to deform in response to a difference in force on the first output shaft and the second output shaft; and
in response to the difference in force on the first output shaft and the second output shaft meeting a threshold, the flexure element is configured to prevent movement of the first phalange with respect to the second phalange.

19. The prosthetic finger of claim 16, wherein:
the flexure element defines one or more openings;
the first output shaft defines:
a central shaft; and
one or more cams;
the second output shaft is positioned at least partially over the central shaft; and
the one or more cams are positioned at least partially within the one or more openings.

20. The prosthetic finger of claim 19, wherein the second output shaft can rotate with respect to the first output shaft.
